(19) United States
(12) Patent Application Publication
(10) Pub. No.: US 2004/0166509 A1
(43) Pub. Date: Aug. 26, 2004

(54) Detecting hormonally active compounds

(76) Inventors: Nancy D. Denslow, Gainesville, FL (US); Patrick M. Larkin, Gainesville, FL (US); Leroy C. Folmar, Gainesville, FL (US); Iris Knoebl, Cincinnati, OH (US); Tara Sabo-Attwood, Ferrisburg, VT (US); Jannet Kocerha, Gainesville, FL (US); Jason Blum, Gainesville, FL (US)

Correspondence Address: Akerman Senterfitt, Suite 400, 222 Lakeview Avenue, West Palm Beach, FL, 33402-3188, US

(21) Appl. No.: 10/663,561

(22) Filed: Sep. 15, 2003

Related U.S. Application Data
(60) Provisional application No. 60/410,414, filed on Sep. 13, 2002.

(51) Int. Cl. C12Q 1/68

(52) U.S. Cl. 435/6; 435/325

(57) ABSTRACT

Purified nucleic acids that are responsive to estrogenic or androgenic agents and derived from fish have been cloned and sequenced. cDNAs that correspond to these nucleic acids are placed on an array containing a set of control genes. Using labeled cDNA probes generated from RNA isolated from control fish and fish exposed to estrogenic compounds, expression levels of the genes responsive to estrogenic compounds are measured. The arrays are useful for monitoring the presence of estrogenic contaminants that are hormonally active in the environment, as well as for screening compounds with estrogenic activity.

### CROSS-REFERENCE TO RELATED APPLICATIONS

The present application claims the priority of U.S. provisional patent application No. 60/410,414 filed on Sep. 13, 2002.

### SEQUENCE LISTING

The present application contains a sequence listing on compact disc which is hereby incorporated herein by reference. The sequence listing file is entitled 5853-238.ST25.txt, contains 427 kilobytes and was created Sep. 15, 2003.

### STATEMENT AS TO FEDERALLY SPONSORED RESEARCH

The invention was made with U.S. government support under grant number P42 ES07375 awarded by the National Institute of Environmental Health Sciences, and grant numbers CR826357-01-0, ID-5267-NTEX, and OD-5378-NTGX awarded by the Environmental Protection Agency. The U.S. government may have certain rights in the invention.

### FIELD OF THE INVENTION

The invention relates to the fields of molecular genetics, endocrinology, and toxicology. More particularly, the invention relates to compositions and methods for detecting androgenic/estrogenic agents in the environment and screening candidate agents for androgenic/estrogenic activity.

### BACKGROUND OF THE INVENTION

The last decade saw the emergence of the field of endocrine disruption after it was discovered that a variety of anthropogenic chemicals act as weak estrogens. Through their interaction with estrogen receptors (ERs), these endocrine-disrupting compounds (EDCs) can alter normal expression of gene products and proteins at critical times during development and reproduction. Environmental contamination with EDCs is therefore a serious concern.

In an effort to detect EDCs in environmental samples, a number of methods have been developed including both in vitro and in vivo assays. Available in vitro assays include those based on hormone receptor-ligand binding, cell proliferation, and reporter gene expression. Although these are relatively inexpensive and amenable to high throughput applications, they provide only limited information about how EDCs affect animals in the environment (see, e.g., Zacharewski T. Environ. Sci. Technol. 31:600-623, 1997; Baker V. A. Toxicol In vitro 15:413-419, 2001). In vivo exposure assays, on the other hand, provide useful information about whole animal responses to EDCs, but can be more cumbersome and expensive than in vitro assays. Moreover, such assays do not provide information about the molecular mechanisms underlying EDC-mediated changes in the animals.

### SUMMARY

The invention is based on the discovery of a large number of sheepshead minnow (SHM) and largemouth bass (LMB) genes that are up-regulated or down-regulated in tissues that have been exposed to an estrogenic or androgenic agent. Thus, whether an environmental sample contains an estrogenic or androgenic agent can be determined by examining a fish (or biological sample obtained from the fish) that was exposed to the sample (e.g., a lake or river) for modulation of expression of these genes. A finding that these genes were modulated in the test fish compared to a control fish not exposed to the sample (or an estrogenic or androgenic agent) indicates that the sample contains an estrogenic or androgenic agent. It was also discovered that different classes of estrogenic or androgenic agents modulated expression of the genes in different patterns depending on the class or mechanism of action of the estrogenic or androgenic agent. Thus, the invention can be used to discern that a particular type of estrogenic or androgenic agent is present in the sample. Based on these discoveries, a screening assay to characterize an unknown molecule's hormonal (e.g., estrogenic or androgenic) activity was developed wherein a fish, fish tissue or fish cell is exposed to a test substance and the effect of the substance on gene expression is compared to known patterns of gene up- or down-regulation. On this basis, the agent can be classified as estrogenic or androgenic and is thus determined to be hormonally active.

Accordingly, the invention features a method for detecting the presence of an agent having estrogenic or androgenic activity in a sample (e.g., a water sample). The method includes the steps of: (A) providing at least one (e.g., at least 2, 3, 4, 5, 10, 25, 100) fish cell which was exposed to the sample; (B) analyzing the at least one fish cell for expression of at least one gene wholly or partially encoded by a nucleotide sequence of SEQ ID NOs: 1-560; and (C) comparing the expression of the at least one gene in the cell compared to the expression of the at least gene in a control cell not exposed to the sample or an agent having estrogenic or androgenic activity. A difference in the expression of the at least one gene in the at least one fish cell compared to the expression of the at least one gene in the control cell indicates that the sample contains an agent having estrogenic or androgenic activity.

In the method, the fish cell can be a large mouth bass cell or a sheep's head minnow cell. It can also be one obtained from a fish that had been exposed to the sample.

Also in the method, the step of analyzing the at least one fish cell for expression of at least one gene (e.g., at least 2, 3, 4, 5, 10, 25, 100) might involve isolating RNA transcripts from the at least one cell, and the step of analyzing the at least one fish cell for expression of at least one gene can include contacting the isolated RNA transcripts or nucleic acids derived therefrom using the isolated RNA transcripts as templates with at least one probe (e.g., at least 2, 3, 4, 5, 10, 25, 100) that hybridizes under stringent hybridization conditions to at least one nucleotide sequence of SEQ ID NOs: 1-560.

The probe can be immobilized on a substrate such as nylon, nitrocellulose, glass, and plastic. It can be on conjugated with a detectable label. In one variation of the method of the invention, the isolated RNA transcripts or nucleic acids derived therefrom are conjugated with a detectable label.

The method of the invention might also include analyzing the control cell not exposed to the sample or an agent having estrogenic or androgenic activity for expression of at least one gene wholly or partially encoded by a nucleotide sequence of SEQ ID NOs: 1-560. In this version of the method, the step of analyzing the control cell for expression of at least one gene can include isolating RNA transcripts from the control cell and contacting the isolated RNA transcripts or nucleic acids derived therefrom using the isolated RNA transcripts as templates with at least one probe (e.g., at least 2, 3, 4, 5, 10, 25, 100) that hybridizes under stringent hybridization conditions to at least one nucleotide sequence (e.g., at least 2, 3, 4, 5, 10, 25, 100) of SEQ ID NOs: 1-560. Also in this version of the method, the RNA transcripts or nucleic acids derived therefrom isolated from the at least one fish cell can be conjugated with a first detectable label and the RNA transcripts or nucleic acids derived therefrom isolated from the control cell are conjugated with a second detectable label differing from the first detectable label.

For example, the method can include isolating RNA transcripts from the at least one fish cell and contacting the RNA transcripts isolated from the at least one fish cell or nucleic acids derived therefrom using the RNA transcripts isolated from the at least one fish cell as templates with at least one molecule that hybridizes under stringent conditions to at least one nucleotide sequence of SEQ ID NOs: 1-560. The at least one probe can be conjugated with a first detectable label and the at least one molecule can be conjugated with a second detectable label differing in chemical structure from the first detectable label. The step of comparing the expression of the at least one nucleic acid in the cell compared to the expression of the at least one nucleic acid in a control cell not exposed to the sample or an agent having estrogenic or androgenic activity may be performed by quantifying the amount of first detectable label associated with the RNA transcripts isolated from the control cell or nucleic acids derived therefrom, and quantifying the amount of second detectable label associated with the RNA transcripts isolated from the at least one fish cell or nucleic acids derived therefrom.

An additional variation of the method of the invention also includes the steps of contacting the fish with the sample; and isolating the at least one fish cell from the fish contacted with the sample.

In another aspect, the invention features a method for determining whether an agent has estrogenic, anti-estrogenic, androgenic or anti-androgenic activity. This method includes the steps of: providing at least one fish cell; contacting the at least one fish cell with the agent; analyzing the at least one fish cell for expression of at least one gene wholly or partially encoded by a nucleotide sequence of SEQ ID NOs: 1-560; and comparing the expression of the at least one gene in the cell compared to the expression of the at least one nucleic acid in a control cell not exposed to the sample or an agent having estrogenic or androgenic activity. A difference in the expression of the at least one nucleic acid in the at least one fish cell compared to the expression of the at least one nucleic acid in the control cell indicates that the agent has estrogenic, anti-estrogenic, androgenic, or anti-androgenic activity.

Yet another aspect of the invention is a substrate having immobilized thereon at least one (e.g., at least 2, 3, 4, 5, 10, 25, 100) nucleic acid comprising a nucleotide sequence of SEQ ID NOs: 1-560 and complements thereof.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: N.Y., 1991; and Lewin, Genes V, Oxford University Press: New York, 1994. Commonly understood definitions of microbiology can be found in Singleton and Sainsbury, Dictionary of Microbiology and Molecular Biology, 3rd edition, John Wiley & Sons: New York, 2002.

By the term “gene” is meant a nucleic acid molecule that codes for a particular protein, or in certain cases a functional or structural RNA molecule.

As used herein, a “nucleic acid” or a “nucleic acid molecule” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced by polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

As used herein, “protein” or “polypeptide” are used synonymously to mean any peptide-linked chain of amino acids, regardless of length or post-translational modification, e.g., glycosylation or phosphorylation.

By the term “estrogenic” is meant acting to produce the effects of an estrogen. An “estrogenic agent” and an “estrogen mimic” is a substance that acts to produce the effects of an estrogen.

As used herein the term “androgenic” means acting to produce the effects of an androgen. An “androgenic agent” and an “androgen mimic” is a substance that acts to produce the effects of an androgen.

When referring to hybridization of one nucleic acid to another, “low stringency conditions” means in 10% formamide, 5× Denhardt's solution, 6×SSPE, 0.2% SDS at 42° C., followed by washing in 1×SSPE, 0.2% SDS, at 50° C.; “moderate stringency conditions” means in 50% formamide, 5× Denhardt's solution, 5×SSPE, 0.2% SDS at 42° C., followed by washing in 0.2×SSPE, 0.2% SDS, at 65° C.; and “high stringency conditions” means in 50% formamide, 5× Denhardt's solution, 5×SSPE, 0.2% SDS at 42° C., followed by washing in 0.1×SSPE, and 0.1% SDS at 65° C. The phrase “stringent hybridization conditions” means low, moderate, or high stringency conditions.

Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

FIG. 1 is a series of macroarrays demonstrating gene expression profiles from SHM exposed to E₂, 17α-ethynyl estradiol (EE₂), diethylstilbestrol (DES), para-nonylphenol (pNP), methoxychlor (MXC), endosulfan (ES) or untreated control fish. Three separate fish were used for each treatment.

FIG. 2 is two graphs showing quantification of the E₂, EE₂, DES, pNP, MXC, ES and control arrays for SHM. Panel A is a plot of the mean±SEM intensity values for each of the cDNA clones arranged in order of their expression. Panel B is a plot of the mean intensity values for each of the cDNA clones for E₂, EE₂, DES, pNP, ES, or MXC divided by the mean intensity values of the respective cDNA clones for untreated control fish. Any clones above the line labeled 1.66 were considered up-regulated genes, any clones below the line labeled 0.42 were considered down-regulated genes, and any clones between these lines were considered constitutive. Genes on the macroarray were designated as constitutive if their intensity values fell within the range of the mean plus one standard deviation of the highest and lowest values of the 11 clones that were used to normalize the data.

FIG. 3 is a series of graphs plotting the quantification of the EE₂ dose response arrays for SHM. Each graph contains a plot of a gene whose expression levels significantly changed more then 2-fold at one or more of the three EE₂ concentrations compared to controls as revealed by one way analysis of variance (P<0.05). AMBP=alpha-1-microglobulin/bikunin precursor protein. The data on both axes are plotted using a log₁₀ scale.

FIG. 4 shows arrays on a plot for control and E₂-treated fish and the results from the array analysis. Panels A and B are arrays that were hybridized with RNA from control (triethylene glycol (TEG)-treated) and E₂-treated SHMs, respectively. Panel C is a plot of the mean intensity value of each cDNA clone on the E₂-treated blots (N=3) over the mean intensity value of each cDNA clone on the control (TEG treated) blots (N=2). The black circles in panel C represent the 17 cDNA clones that were identified by DD analysis to be constitutive. Any clones above the dotted gray line labeled 1.27 were considered E₂ up-regulated genes, any clones below the dotted gray line labeled 0.83 were considered E₂ down-regulated genes, and any clones between the two gray dotted lines were considered constitutive genes. In panel C, a is transferrin, b is vitellogenin (Vtg ) β, c is ZP2, and d is vitellogenin α. There is a break in the graph of panel C from 2 to 10 log (intensity) units.

FIG. 5 is two graphs showing gene expression profiles from control and E₂-treated male LMB. (A) shows the mean±SEM intensity values for each of the cDNA clones arranged in order of their expression (black circles are E₂, gray circles are control); (B) illustrates the mean intensity values for each of the cDNA clones for E₂ divided by the mean intensity values of the respective cDNA clones from control fish. Any genes outside of the upper and lower solid gray lines in the figure change by more then two-fold and are considered to be up or down-regulated. Genes that exhibited a significant change in expression at P<0.05 are shown by a double asterisk; whereas genes that exhibited a significant change in expression at P<0.1 are shown by a single asterisk (t-tests). Three separate fish were used for each treatment. Only genes that were found in at least one of the treatments to be at least three standard deviations from the mean of the 12 ribosomal protein (r-protein) genes used to normalize the data (0.98±0.41) are plotted. AR=androgen receptor, ER=estrogen receptor, and NADH=Nicotinamide Adenine Dinucleotide (reduced form).

FIG. 6 is two graphs showing gene expression profiles from control and 4-NP-treated male LMB. The order of genes in this figure corresponds to the order in FIG. 5.

FIG. 7 is two graphs showing gene expression profiles from control and p, p′-DDE treated male LMB. The order of genes in this figure corresponds to the order in FIG. 5.

FIG. 8 is two graphs showing gene expression profiles from control and p, p′-DDE treated female LMB. The order of genes in this figure corresponds to the order in FIG. 5.

FIG. 9 is a list of genes whose expression is increased or decreased more than two-fold following exposure of LMB to E₂, 4-NP, and p,p′-DDE.

### DETAILED DESCRIPTION

The invention is premised in part on the discovery of nucleic acids (e.g., those of SEQ ID NOs: 1-560) whose expression is modulated in response to estrogenic/androgenic agents in fish such as SHM and LMB. Several of these nucleic acids were not previously characterized. Thus, the invention includes these nucleic acids, variants of these nucleic acids, proteins encoded by these nucleic acids, antibodies against these proteins, as well as other embodiments that can be made by one of skill in the art having knowledge of these sequences. An important application of the discovery is an assay for detecting modulation of expression of these nucleic acids in order to analyze an environmental sample or uncharacterized sample molecule. Detection of such modulation in a biological sample indicates that the sample or molecule exerts a hormonal activity (e.g., estrogenic or androgenic activity) or an anti-hormonal activity (e.g., anti-estrogenic, anti-androgenic activity).

The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Various techniques using PCR are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, ©1991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

### Novel Fish Genes

As several new genes were identified and characterized in making the invention, the invention provides several purified nucleic acids from SHM and LMB that are modulated in response to androgenic/estrogenic compounds. SHM nucleic acids of the invention have the nucleotide sequences of SEQ ID NOs: 151-419, while LMB nucleic acids of the invention have the nucleotide sequences of SEQ ID NOs: 1-150, 420-560.

Various assays described herein include a step of analyzing expression of a SHM or LMB gene modulated in response to an estrogenic or androgenic agent. Thus, polynucleotides that preferentially bind to nucleic acids encoded by the gene (e.g., mRNA, cDNA, DNA complements of cDNA, etc.) are also within the invention. Such polynucleotides can have the exact sequence of all or a portion of SEQ ID NOs: 1-560 or the complements of SEQ ID NOs: 1-560. Because hybridization of two nucleic acids does not generally require 100% complementarity, variants of such polynucleotides are also within the invention. These might include naturally occurring allelic variants of native LMB or SHM nucleic acids or non-naturally occurring variants that show sequence similarity to all or portions of SEQ ID NOs: 1-560 or the complements of SEQ ID NOs: 1-560

Naturally occurring allelic variants of native LMB or SHM nucleic acids within the invention are nucleic acids isolated from LMB and SHM that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with native LMB and SHM nucleic acids, and encode polypeptides having at least one functional activity in common with LMB and SHM polypeptides. Homologs of native LMB and SHM nucleic acids within the invention are nucleic acids isolated from other species (e.g., other fish species) that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with native LMB and SHM nucleic acids, and encode polypeptides having at least one functional activity in common with native LMB and SHM polypeptides. Naturally occurring allelic variants of LMB and SHM nucleic acids and homologs of LMB and SHM nucleic acids can be isolated by using a library screen, other assays described herein, or other techniques known in the art. The nucleotide sequence of such homologs and allelic variants can be determined by conventional DNA sequencing methods. Alternatively, public or non-proprietary nucleic acid databases can be searched to identify other nucleic acid molecules (e.g., nucleic acids from other species) having a high percent (e.g., 70, 80, 90% or more) sequence identity to native LMB and SHM nucleic acids.

Non-naturally occurring LMB and SHM nucleic acids variants are nucleic acids that do not occur in nature (e.g., are made by the hand of man), have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with native LMB and SHM nucleic acids, and encode polypeptides having at least one functional activity in common with native LMB and SHM polypeptides. Examples of non-naturally occurring LMB and SHM nucleic acids are those that encode a fragment of an LMB or SHM protein, those that hybridize to native LMB and SHM nucleic acids or a complement of native LMB and SHM nucleic acids under stringent conditions, those that share at least 65% sequence identity with native LMB and SHM nucleic acids or a complement of native LMB and SHM nucleic acids, and those that encode an LMB or SHM fusion protein.

Nucleic acids encoding fragments of LMB and SHM polypeptides within the invention are those that encode, e.g., 2, 5, 10, 25, 50, 100, 150, 200, 250, 300, or more amino acid residues of LMB or SHM polypeptides. Shorter oligonucleotides (e.g., those of 6, 12, 20, 30, 50, 100, 125, 150 or 200 base pairs in length) that encode or hybridize with nucleic acids that encode fragments of LMB or SHM polypeptides can be used as probes, primers, or antisense molecules. Longer polynucleotides (e.g., those of 300, 400, 500, 600, 700, 800, 900, 1000, 1100, 1200 or 1300 base pairs) that encode or hybridize with nucleic acids that encode fragments of LMB or SHM polypeptides can be used in place of native LMB or SHM polynucleotides in applications where it is desired to modulate a functional activity of native LMB or SHM polypeptides. Nucleic acids encoding fragments of LMB or SHM polypeptides can be made by enzymatic digestion (e.g., using a restriction enzyme) or chemical degradation of full length LMB or SHM nucleic acids or variants of LMB or SHM nucleic acids.

Nucleic acids that hybridize under stringent conditions to the nucleic acid of SEQ ID NOs: 1-560 or the complement of SEQ ID NOs: 1-560 are also within the invention. For example, nucleic acids that hybridize to SEQ ID NOs: 1-560 or the complement of SEQ ID NOs: 1-560 under low stringency conditions, moderate stringency conditions, or high stringency conditions are within the invention. Preferred such nucleic acids are those having a nucleotide sequence that is the complement of all or a portion of SEQ ID NOs: 1-560. Other variants of LMB or SHM nucleic acids within the invention are polynucleotides that share at least 65% (e.g., 65, 70, 75, 80, 85, 90, 91, 92, 93, 94, 95, 96, 97, 98, and 99%) sequence identity to SEQ ID NOs: 1-560 or the complement of SEQ ID NOs: 1-560. Nucleic acids that hybridize under stringent conditions to or share at least 65% sequence identity with SEQ ID NOs: 1-560 or the complement of SEQ ID NOs: 1-560 can be obtained by techniques known in the art such as by making mutations in native LMB or SHM nucleic acids, by isolation from an organism expressing such a nucleic acid (e.g., a fish expressing a variant of native LMB or SHM nucleic acids), or an organism other than a fish expressing a homolog of native LMB or SHM nucleic acids.

Nucleic acid molecules of the present invention may be in the form of RNA or in the form of DNA (e.g., cDNA, genomic DNA, and synthetic DNA). The DNA may be double-stranded (ds) or single-stranded (ss), and if single-stranded may be the coding (sense) strand or non-coding (anti-sense) strand. The nucleic acid molecules of the present invention may also be polynucleotide analogues such as peptide nucleic acids (PNA). See, e.g. Gambari R., Curr. Pharm. Des. 7:1839-1862, 2001; U.S. Pat. No. 6,395,474; and PCT patent application publication number WO 86/05518. The sequences which encode native LMB and SHM gene products may be identical to the nucleotide sequences shown in SEQ ID NOs:1-560. They may also be different sequences which, as a result of the redundancy or degeneracy of the genetic code, encode the same polypeptides as the polynucleotides of SEQ ID NOs:1-560. Other nucleic acid molecules within the invention are variants of nucleic acids of SEQ ID NOs: 1-560 such as those that encode fragments, analogs and derivatives of native proteins encoded by nucleic acids of SEQ ID NOs: 1-560. Such variants may be, e.g., a naturally occurring allelic variant of native nucleic acids of SEQ ID NOs: 1-560, a homolog of native nucleic acids of SEQ ID NOs:1-560, or a non-naturally occurring variant of native nucleic acids of SEQ ID NOs: 1-560. These variants have a nucleotide sequence that differs from native nucleic acids of SEQ ID NOs: 1-560 in one or more bases. For example, the nucleotide sequence of such variants can feature a deletion, addition, or substitution of one or more nucleotides of native nucleic acids of SEQ ID NOs: 1-560. Nucleic acid insertions are preferably of about 1 to 10 contiguous nucleotides, and deletions are preferably of about 1 to 30 contiguous nucleotides.

### Probes and Primers

Nucleic acids that hybridize under stringent conditions to the nucleic acid sequences of SEQ ID NOs: 1-560 or the complement of the nucleic acid sequences of SEQ ID NOs: 1-560 can be used in the invention. For example, such nucleic acids can be those that hybridize to the nucleic acid sequences of SEQ ID NOs: 1-560 or the complement of the nucleic acid sequences of SEQ ID NOs: 1-560 under low stringency conditions, moderate stringency conditions, or high stringency conditions. Preferred such nucleic acids are those having a nucleotide sequence that is the complement of all or a portion of a nucleic acid sequence of SEQ ID NOs: 1-560. Others that might be used include polynucleotides that share at least 65% (e.g., 65, 70, 75, 80, 85, 90, 91, 92, 93, 94, 95, 96, 97, 98, and 99%) sequence identity to a native nucleic acid sequence of SEQ ID NOs: 1-560 or the complement of a native nucleic acid sequence of SEQ ID NOs: 1-560. Nucleic acids that hybridize under stringent conditions to or share at least 65% sequence identity with the nucleic acid sequences of SEQ ID NOs: 1-560 or the complement of the nucleic acid sequences of SEQ ID NOs: 1-560 can be obtained by techniques known in the art such as by making mutations in a native nucleic acid sequence of SEQ ID NOs: 1-560, or by isolation from an organism expressing such a nucleic acid (e.g., an allelic variant).

Methods of the invention utilize oligonucleotide probes (i.e., isolated nucleic acid molecules conjugated with a detectable label or reporter molecule, e.g., a radioactive isotope, ligand, chemiluminescent agent, or enzyme); and oligonucleotide primers (i.e., isolated nucleic acid molecules that can be annealed to a complementary target DNA strand by nucleic acid hybridization to form a hybrid between the primer and the target DNA strand, then extended along the target DNA strand by a polymerase, e.g., a DNA polymerase). Primer pairs can be used for amplification of a nucleic acid sequence, e.g., by the PCR or other conventional nucleic-acid amplification methods.

PCR primers can be used to amplify the nucleic acid sequences of SEQ ID NOs: 1-560 using known PCR and RT-PCR protocols. Such primers can be designed according to known methods as PCR primer design is generally known in the art. See, e.g., methodology treatises such as Basic Methods in Molecular Biology, 2nd ed., ed. Davis et al., Appleton & Lange, Norwalk, CN, 1994; and Molecular Cloning: A Laboratory Manual, 3rd ed., vol.1-3, ed. Sambrook et al., Cold Spring Harbor Laboratory Press, Cold Spring Harbor, N.Y., 2001.

Probes and primers utilized in methods of the invention are generally 15 nucleotides or more in length, preferably 20 nucleotides or more, more preferably 25 nucleotides, and most preferably 30 nucleotides or more. Preferred probes and primers are those that hybridize to a native nucleic acid sequence of SEQ ID NOs: 1-560 (or cDNA or mRNA) sequence under high stringency conditions, and those that hybridize to homologs of the nucleic acid sequences of SEQ ID NOs: 1-560 under at least moderately stringent conditions. Preferably, probes and primers according to the present invention have complete sequence identity with a native nucleic acid sequence of SEQ ID NOs: 1-560. However, probes differing from this sequence that retain the ability to hybridize to a native nucleic acid sequence of SEQ ID NOs: 1-560 under stringent conditions may be designed by conventional methods and used in the invention. Primers and probes based on the nucleic acid sequences of SEQ ID NOs: 1-560 disclosed herein can be used to confirm (and, if necessary, to correct) the disclosed nucleic acid sequences of SEQ ID NOs: 1-560 by conventional methods, e.g., by re-cloning and sequencing a native nucleic acid sequence of SEQ ID NOs: 1-560 or cDNA corresponding to a native nucleic acid sequence of SEQ ID NOs: 1-560.

### Proteins Encoded by Nucleic Acid Sequences of SEQ ID NOs: 1-560

The invention also provides polypeptides encoded in whole or in part by the nucleic acid sequences of SEQ ID NOs: 1-560. Some polypeptides encoded by the nucleic acids of SEQ ID NOs: 1-560 are expressed at higher levels when the nucleic acids are exposed to hormonal compounds compared to control nucleic acids not exposed to the hormonal compound. Other polypeptides encoded in whole or in part by the nucleic acid sequences of SEQ ID NOs: 1-560 are expressed at lower levels when exposed to hormonal compounds compared to the expression of nucleic acids not exposed to the hormonal compound.

Variants of native proteins encoded in whole or in part by nucleic acid sequences of SEQ ID NOs: 1-560 such as fragments, analogs and derivatives of native proteins encoded by nucleic acid sequences of SEQ ID NOs: 1-560 may also be used in methods of the invention. Such variants include, e.g., a polypeptide encoded in whole or in part by a naturally occurring allelic variant of a native nucleic acid sequence of SEQ ID NOs: 1-560, a polypeptide encoded by an alternative splice form of a native nucleic acid sequence of SEQ ID NOs: 1-560, a polypeptide encoded in whole or in part by a homolog of a native nucleic acid sequence of SEQ ID NOs: 1-560, and a polypeptide encoded in whole or in part by a non-naturally occurring variant of a native nucleic acid sequence of SEQ ID NOs: 1-560.

Protein variants encoded by a sequence having homology to a nucleic acid sequence of SEQ ID NOs: 1-560 have a peptide sequence that differs from a native protein encoded in whole or in part by a nucleic acid sequence of SEQ ID NOs: 1-560 in one or more amino acids. The peptide sequence of such variants can feature a deletion, addition, or substitution of one or more amino acids of a native polypeptide encoded in whole or in part by a nucleic acid sequence of SEQ ID NOs: 1-560. Amino acid insertions are preferably of about 1 to 4 contiguous amino acids, and deletions are preferably of about 1 to 10 contiguous amino acids. In some applications, variant proteins substantially maintain a native nucleic acid sequence of SEQ ID NOs: 1-560-encoded protein functional activity. For other applications, variant proteins lack or feature a significant reduction in a nucleic acid sequence of SEQ ID NOs: 1-560-encoded protein functional activity. Where it is desired to retain a functional activity of a native protein encoded in whole or in part by a nucleic acid sequence of SEQ ID NOs: 1-560, preferred protein variants can be made by expressing nucleic acid molecules within the invention that feature silent or conservative changes. Variant proteins with substantial changes in functional activity can be made by expressing nucleic acid molecules within the invention that feature less than conservative changes.

Nucleic acid sequences of SEQ ID NOs: 1-560-encoded protein fragments corresponding to one or more particular motifs and/or domains or to arbitrary sizes, for example, at least 5, 10, 25, 50, 75, 100, 125, 150, 175, 200, and 250 amino acids in length may be utilized in methods of the present invention. Isolated peptidyl portions of proteins encoded by a nucleic acid sequence of SEQ ID NOs: 1-560 can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a protein encoded by a nucleic acid sequence of SEQ ID NOs: 1-560 used in methods of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a native nucleic acid sequence of SEQ ID NOs: 1-560-encoded protein.

Methods of the invention may also involve recombinant forms of the nucleic acid sequences of SEQ ID NOs: 1-560-encoded proteins. Recombinant polypeptides preferred by the present invention, in addition to native nucleic acid sequence of SEQ ID NOs: 1-560-encoded protein, are encoded by a nucleic acid that has at least 85% sequence identity (e.g., 85, 86, 87, 88, 89, 90, 91, 92, 93, 94, 95, 96, 97, 98, 99, 100%) with a native nucleic acid sequence of SEQ ID NOs: 1-560. In a preferred embodiment, variant proteins lack one or more finctional activities of native nucleic acid sequence of SEQ ID NOs: 1-560-encoded protein.

Protein variants can be generated through various techniques known in the art. For example, protein variants can be made by mutagenesis, such as by introducing discrete point mutation(s), or by truncation. Mutation can give rise to a protein variant having substantially the same, or merely a subset of the functional activity of a native protein encoded in whole or in part by a nucleic acid sequence of SEQ ID NOs: 1-560. Alternatively, antagonistic forms of the protein can be generated which are able to inhibit the function of the naturally occurring form of the protein, such as by competitively binding to another molecule that interacts with a protein encoded in whole or in part by a nucleic acid sequence of SEQ ID NOs: 1-560. In addition, agonistic forms of the protein may be generated that constitutively express one or more nucleic acid sequence of SEQ ID NOs: 1-560-encoded protein functional activities. Other protein variants that can be generated include those that are resistant to proteolytic cleavage, as for example, due to mutations that alter protease target sequences. Whether a change in the amino acid sequence of a peptide results in a protein variant having one or more functional activities of a native nucleic acid sequence of SEQ ID NOs: 1-560-encoded protein can be readily determined by testing the variant for a native nucleic acid sequence of SEQ ID NOs: 1-560-encoded protein functional activity.

### Antibodies

Antibodies that specifically bind nucleic acid sequence of SEQ ID NOs: 1-560-encoded proteins can be used in methods of the invention, for example, in the detection of nucleic acid sequence of SEQ ID NOs: 1-560-encoded protein expression. Antibodies of the invention include polyclonal antibodies and, in addition, monoclonal antibodies, single chain antibodies, Fab fragments, F(ab′)₂ fragments, and molecules produced using a Fab expression library. Antibodies can be of any immunoglobulin class including IgG, IgM, IgE, IgA, IgD and any subclass thereof.

Antibodies that specifically recognize and bind to nucleic acid sequence of SEQ ID NOs: 1-560-encoded proteins are useful in methods of the present invention. For example, such antibodies can be used in an immunoassay to monitor the level of the corresponding protein produced by a cell or an animal (e.g., to determine the amount or subcellular location of a nucleic acid sequence of SEQ ID NOs: 1-560-encoded protein). Methods of the invention may also utilize antibodies, for example, in the detection of a nucleic acid sequence of SEQ ID NOs: 1-560-encoded protein in an environmental sample. Antibodies also can be used in a screening assay to measure the effect of a candidate agent on expression or localization of a nucleic acid sequence of SEQ ID NOs: 1-560-encoded protein.

### Detecting the Presence of an Agent Having Androgenic/estrogenic Activity

Within the invention, SEQ ID NOs:1-560 are used in various methods for detecting the presence of estrogenic/androgenic agents (e.g., EDCs) such as E₂, EE₂, DES, MXC, ES, 4-NP, p-chlorophenyl, and p,p′-DDE in a sample. Examples of other EDCs that may be detected using compositions and methods of the invention include benzenehexachloride, 1,2-dibromoethane, chloroform, dioxins, furans, octachlorostyrene, PBBs, PCBs, PCB, hydroxylated PBDEs, and pentachlorophenol as well as others disclosed in Hormonally Active Agents In The Environment, Ed. by The Committee On Hormonally Active Agents In The Environment Board On Environmental Studies and Toxicology Commission On Life Sciences And National Research Council, National Academy Press, Washington D.C., 1999.

Methods for detecting the presence of an agent having estrogenic or androgenic activity in a sample involve a first step of providing at least one fish cell which was exposed to the sample. A fish cell of the invention can be a cell from any fish, preferably a cell from a SHM or LMB. A sample can be obtained from a number of sources, including a body of water (e.g., river, lake, stream, canal, estuary, pond, etc.) as well as sediment obtained from a body of water or from a site near or contacting a body of water (e.g., sediment from a lake or river bed). The fish cell exposed to the sample can be a cell taken from a fish that was present in a body of water (or in contact with sediment) from which the sample (i.e., environmental sample) was taken. The fish cell can also be a cell isolated from a provided fish that was contacted with the sample (e.g., taken from a fish that was exposed to a sample in controlled, laboratory conditions). Alternatively, the fish cell can be one that was cultured and exposed to the sample in vitro.

A second step of this method involves analyzing the at least one fish cell for expression of at least one gene encoded by a nucleotide sequence selected from the group consisting of SEQ ID NOs: 1-560. A number of methods for analyzing gene expression are described below. A third step of this method involves comparing the expression of the at least one gene in the cell compared to the expression of the at least one gene in a control cell not exposed to the sample or an agent having estrogenic or androgenic activity, wherein a difference in the expression of the at least one gene in the at least one fish cell compared to the expression of the same at least one nucleic acid in the control cell indicates that the sample contains an agent having estrogenic or androgenic activity.

The step of analyzing the at least one fish cell can include analyzing the cell for expression of at least two (e.g., 2, 3, 4, 5, 6, 7, 8, 9, 10, 15, 20, 25, 100) different genes, each being wholly or partially encoded by a nucleotide sequence selected from the group consisting of SEQ ID NOs: 1-560. To analyze the cell for expression of at least one gene, RNA transcripts can be isolated from the at least one cell. The isolated RNA transcripts or nucleic acids derived therefrom can be used as templates and contacted with at least one probe that hybridizes under stringent conditions to at least one nucleotide sequence selected from the group consisting of SEQ ID NOs: 1-560. This step can also include contacting the RNA transcripts or nucleic acids derived therefrom with at least two (e.g., 2, 3, 4, 5, 6, 7, 8, 9, 10, 15, 20, 25, 50, 75, 100, 150) different probes that each hybridize under stringent conditions to a different nucleotide sequence selected from the group consisting of SEQ ID NOs: 1-560. The at least one probe (or probes) or the isolated RNA transcripts (or nucleic acids derived therefrom) can be conjugated with a detectable label such as a fluorphore or a radioactive molecule or compound. The probe(s) can be immobilized on a substrate (e.g., array) before placed in contact with RNA transcripts isolated from a fish cell or control cell, or can be contacted with the RNA transcripts in solution (e.g., real-time PCR assay) rather than in the presence of a substrate. Examples of substrates that may be used include nylon, nitrocellulose, glass, and plastic.

In another method of detecting the presence of an agent having estrogenic or androgenic activity in a sample, the control cell not exposed to the sample or an agent having estrogenic or androgenic activity is analyzed for expression of at least one gene encoded by a nucleotide sequence selected from the group consisting of SEQ ID NOs: 1-560. For example, RNA transcripts can be isolated from the control cell and contacted with the RNA transcripts or nucleic acids derived therefrom using the isolated RNA transcripts as templates with at least one probe that hybridizes under stringent conditions to at least one nucleotide sequence selected from the group consisting of SEQ ID NOs: 1-560. This method can further include isolating RNA transcripts from the at least one fish cell and contacting the RNA transcripts isolated from the at least one fish cell (or nucleic acids derived therefrom) with at least one molecule that hybridizes under stringent conditions to at least one nucleotide sequence selected from the group consisting of SEQ ID NOs: 1-560. In some applications, the at least one probe is conjugated with a first detectable label and the at least one molecule is conjugated with a second detectable label differing in chemical structure from the first detectable label. In other applications, the RNA transcripts (or nucleic acids derived therefrom) isolated from the at least one fish cell are conjugated with a first detectable label and the RNA transcripts isolated from the control cell are conjugated with a second detectable label differing in chemical structure from the first detectable label.

To compare expression of the at least one nucleic acid in the fish cell compared to the expression of the at least one nucleic acid in a control cell not exposed to the sample or an agent having estrogenic or androgenic activity, both 1) the amount of first detectable label associated with the RNA transcripts isolated from the control cell (or nucleic acids derived therefrom) and 2) the amount of second detectable label associated with the RNA transcripts isolated from the at least one fish cell (or nucleic acids derived therefrom) is quantified.

In one example of comparing expression of the at least one nucleic acid in the fish cell to the expression of the at least one nucleic acid in the control cell, the labeled RNA transcripts (or nucleic acids derived therefrom) isolated from the at least one fish cell and from the control cell are contacted e.g., on an array as described herein. Hybridization of the differentially labeled transcripts to the nucleic acids is then detected (e.g., using an imaging device such as a phosphor screen or autoradiographic film) and signal intensities are quantitatively analyzed (e.g., using a software program such as Atlaslmage™ 2.01 Clontech, Palo Alto, Calif.).

Among the traditional methods that can be employed for gene expression analyses are DD RT-PCR, nucleic acid arrays, quantitative PCR (e.g., real-time PCR), in situ hybridization, serial analysis of gene expression (SAGE), and subtractive hybridization. DD RT-PCR, for example, isolates differentially expressed genes using both arbitrary and anchored oligo-dT primers (Liang & Pardee, 1992; Liang et al., 1994; and Genome Analysis: A Laboratory Manual Series 1, ed: B. Birren et al., 1997, Cold Spring Harbor Laboratory Press, Cold Spring Harbor, N.Y.). A typical DD RT-PCR protocol involves several steps including reverse transcription using anchored oligo-dT primers, amplification of cDNA using one anchored and one arbitrary primer, electrophoresis of PCR products, purification of the product of interest, and cloning and sequencing of the product. In one method, DD-RT-PCR is performed with the RNAimage mRNA Differential Display system (GenHunter; Nashville, Tenn.) using one-base anchored oligo-dT primers (Liang et al., 1994) as described previously (Denslow et al., 1999a; and Denslow et al., 2001).

In vitro quantitation of gene expression can be performed using a number of real-time quantitative PCR assays. Real-time quantitative PCR assays typically involve labeling a target nucleic acid with a first fluorescing dye and labeling a probe with a second fluorescing dye. For example, Multiplex TaqMan® (Applied Biosystems, Foster City, Calif.) assays can be performed using the ABI PRISM® 7700 Sequence Detection System (Applied Biosystems, Foster City, Calif.), capable of detecting multiple dyes with distinct emission wavelengths. Some real-time quantitative PCR applications involve the use of fluorescence resonance energy transfer (FRET) between fluorochromes introduced into DNA molecules (e.g., molecular beacon assays). For a review of FRET techniques, see Vet et al., Expert Rev. Mol. Diagn. 2:77-86, 2002.

A preferred technique for detecting the presence of estrogenic compounds involves the use of nucleic acid arrays. Nucleic acid arrays allow the simultaneous monitoring of expression patterns of multiple genes from the same sample. Arrays are an appropriate tool for rapidly screening large numbers of genes. Examples of nucleic acid arrays include microarrays and macroarrays. Methods involving nucleic acid arrays are reviewed in Ringner et al., Pharmacogenomics 3:403-415, 2002; Epstein et al., Curr. Opin. Biotechnol. 11:36-41, 2000; Granjeaud et al., BioEssays 21:781-790, 1999; Hatakeyama K., Nippon Rinsho 57:465-473, 1999; DNA Microarrays: A Molecular Cloning Manual, ed: D. Bowtell and J. Sambrook, 2002, Cold Spring Harbor Laboratory Press, Cold Spring Harbor, N.Y., and U.S. Pat. No. 6,410,229. The construction and use of nucleic acid arrays containing fish genes is described below.

### Arrays

The nucleic acids (and proteins and antibodies) of the invention are preferably useful for assaying a sample for the presence of a hormonal agent (e.g., an estrogenic, sample in an environmental water sample). In this regard, nucleic acid-based assays are presently preferred. The invention thus provides a substrate having immobilized thereon at least one nucleic acid including a nucleotide sequence selected from the group consisting of SEQ ID NOs: 1-560 and complements thereof. A typical substrate having immobilized thereon at least one nucleic acid of the invention is an array of.fish nucleic acids, including nucleic acids (e.g., genes and gene fragments) responsive to androgenic and estrogenic compounds. Arrays containing fish-derived nucleic acids responsive to androgenic and/or estrogenic compounds can be used in a number of applications. For example, the arrays can be used to monitor the presence and distribution of androgenic and estrogenic contaminants in the environment. The arrays can also be used to screen for synthetic or natural agents having androgenic or estrogenic activity. An example of an array provided by the invention is a macroarray containing LMB- or SHM-derived nucleic acids. On a preferred macroarray of the invention, a minimum number of nucleotides of 150 is included for each nucleic acid (e.g., 2, 10, 50, 75, 100, 125, 130, 135, 140, 145, 150, 155, 160, 165, 170, 200, 250, 300, 350, 400 or more). A portion of the nucleic acids on the macroarray are responsive to estrogenic compounds. A list of nucleic acids that may be contained within a macroarray of the invention is presented in Table III (SHM), Table II (LMB), and Table IV (SHM and LMB).

To construct a cDNA macroarray, cDNA is first prepared from RNA. Techniques for preparing cDNA from RNA are widely known, and are described in methodology treatises such as Sambrook and Russell supra and Ausubel et al., supra. In one example, cDNA clones (e.g., miniprep cDNAs) derived from DD RT-PCR analysis (as described above) are PCR-amplified using primers specific to the cloning vector (e.g., pGEMT-Easy, Promega, Madison, Wis.). Any suitable thermocycling conditions that result in amplification of the desired product may be used. After completion of the PCR, the products are purified (e.g., in a spin-column, Quiagen, Chatsworth, Calif.) and then concentrated (e.g., in a speed-vac). Aliquots of the PCR products are then resolved electrophoretically (e.g., run on a 1.2% agarose gel containing 0.3 mM ethidium bromide). The resultant gels are analyzed (e.g., digitally imaged using a UVP Bio Doc-It camera, Ultra Violet Laboratory Products, Upland Calif.) and the concentration of each PCR product is determined. Typically, concentrations of PCR products are determined by comparing the intensity of each band to a standard curve derived from a low DNA mass ladder (InVitrogen Corporation, Carlsbad, Calif.).

Once the PCR products are purified and their concentrations determined, they are then spotted onto a membrane (e.g., nylon membrane). Methods for spotting cDNAs onto membranes are discussed in Diehl et al., NAR 29:E38, 2001; Shieh et al., Biotechniques 32:1360-1362 & 1364-1365, 2002; and Schuchhardt et al., NAR 28:E47, 2000. In one method of spotting the cDNAs onto a membrane, PCR products are denatured, quenched on ice, and robotically spotted onto nylon membranes (Fisher Scientific). In this method, membranes are cross-linked and stored under vacuum at room temperature until the hybridization step. Various controls are also spotted onto the membranes. These controls provide information about cDNA labeling efficiency, blocking at the pre-hybridization step, and non-specific binding. Any genes that are not responsive to estrogen may be used as negative control genes on an array of the invention. Control genes that are not responsive to estrogen include Arabidopsis thaliana cDNA clones, Cot-1 repetitive sequences, polyA sequence (SpotReport 3, Stratagene, LaJolla, Calif.), and a M13 sequence (vector but no cDNA insert). The consistency of the spotting technique may be assessed by spotting on the array multiple cDNA products from the same gene that were amplified in separate PCR reactions.

For the generation of probes, mRNA from fish exposed to an estrogenic compound (e.g., E₂, EE₂, DES, pNP, ES, MXC) and mRNA from control fish (i.e., fish not exposed to estrogenic compounds), is extracted and purified. mRNA may be purified by a number of known techniques, including the use of affinity columns (Qiagen, Chatsworth, Calif.). In addition to RNA probes, cDNA probes may also be used. The labeling of nucleotide probes is described in Relogio et al., NAR 30:351, 2002; and Yu et al., Mol. Vis. 8:130-137, 2002. Probes may be labeled using any of a number of techniques, including fluorescence (e.g., Atlas Glass Fluorescent Labeling Kit, Clontech, Palo Alto, Calif.), resonance light scattering (Bao et al., Anal. Chem. 74:1792-1797, 2002), gold nanoparticle labeling (Fritzsche et al., J. Biotechnol. 1:37-46, 2001) and radioactive methods. In one example of radiolabeling RNA probes, DNase-treated total RNA from fish is subjected to random primer labeling with α-³³P dATP (Strip-EZ RT, Ambion, Austin, Tex.). RNAs may also be radiolabeled using a kit such as AtlasPure™ RNA Labeling System. Typically, blots are prehybridized for several hours, hybridized overnight with probe-containing solution, and then washed several times.

To detect hybridization of the probe to nucleotides on an array, membranes are exposed to a suitable imaging device, such as a phosphor screen (Molecular Dynamics, Piscataway, N.J.) or autoradiographic film for an appropriate period of time (e.g.,several hours). Signal intensities may be quantitatively analyzed using a suitable software program, such as Atlaslmage™ 2.01 (Clontech, Palo Alto, Calif.). Blots may also be quantitatively evaluated using a Typhoon 8600 imaging system (Molecular Dynamics). For each nucleotide (e.g., cDNA) clone on an array, the general background of each membrane is subtracted from the average value of the duplicate spots on the membrane. The values are normalized to the average value of several (e.g., 11) nucleotide (e.g., cDNA) clones. Gene array data is analyzed using a suitable statistical analysis. For example, linear regression and one-way analysis of variance, with Tukey post-hoc analysis (SigrnaStat and SigmaPlot, Jandel, Calif.) may be used to analyze the gene array data.

### Determining Whether an Agent has Estrogenic, Anti-Estrogenic, Androgenic, or Anti-Androgenic Activity

In addition to detecting the presence of estrogenic compounds in the environment, nucleic acid arrays containing one or more nucleotide sequences of SEQ ID NOs: 1-560 of the invention may also be used to screen for compounds with estrogenic, anti-estrogenic, androgenic, or anti-androgenic activity. Estrogenic compounds (e.g., estrogen, estrogen mimics) have possible uses in a number of disorders, including the treatment of cardiovascular disease, menopausal symptoms and menopausal osteoporosis. Molecules or compounds with anti-estrogenic activity (e.g., flavonoids) have a number of possible applications, including the treatment of breast cancer. Androgenic agents also have a number of applications, including the treatment of sexual dysfunction, depression and pelvic endometriosis. Androgenic agents are also fed to livestock as growth-inducing agents. For the treatment of prostate enlargement and acne, anti-androgenic agents are useful.

A method for determining whether an agent has estrogenic, anti-estrogenic, androgenic, or anti-androgenic activity involves several steps. A first step in this method includes providing at least one fish cell. In a second step of the method, the at least one fish cell is contacted with the agent. In a third step, the at least one fish cell is analyzed for expression of at least one gene wholly or partially encoded by a nucleotide sequence selected from the group consisting of SEQ ID NOs: 1-560. A fourth step of the method includes comparing the expression of the at least one gene in the cell compared to the expression of the at least one nucleic acid in a control cell not exposed to the sample or an agent having estrogenic, anti-estrogenic, androgenic or anti-androgenic activity. In this method, a difference in the expression of the at least one nucleic acid in the at least one fish cell compared to the expression of the at least one nucleic acid in the control cell indicates that the agent has estrogenic, anti-estrogenic, androgenic, or anti-androgenic activity.

In one embodiment of determining if a test agent increases or decreases expression of a gene responsive to estrogen, cells are first exposed to the test agent in vitro. For example, multiple compounds can be tested simultaneously by plating cells in a multi-well plate (e.g., in a 96 well tissue culture plate) and contacting one test compound per well. RNA from the exposed cells as well as from control cells (i.e., negative control cells not exposed to the test compound and positive control cells exposed to the test compound) is isolated and reverse transcribed to cDNAs. The cDNAs are labeled to generate probes as described above, and contacted with the nucleic acid arrays of the invention. Hybridization of the labeled probes to the nucleic acids of the array (e.g., SEQ ID NOs: 1-560) is analyzed as described above. Alternatively, whole fish can be exposed to the test agents in the water or through the food. This allows for normal metabolic processes to occur within the various tissues of the fish to end up with an agent that has either the same or more or less activity then the parent agent.

### EXAMPLES

The present invention is further illustrated by the following specific examples. The examples are provided for illustration only and are not to be construed as limiting the scope or content of the invention in any way.

### Example 1—Expression Profiling of Estrogenic Compounds Using A SHM cDNA Macroarray Methods

Amplification of cDNA to be spotted on macroarrays: Minipreps of 30 cDNA clones derived from DD RT-PCR analysis (Denslow et al., Gen. Comp. Endocrinol. 121:250-260, 2001; Denslow et al., Comp. Biochem. Physiol. B. Biochem. Mol. Biol. 129:277-282, 2001) were PCR amplified in a 300 μL reaction containing 1×PCR Buffer A (Promega, Madison, Wis.), 2 mM MgCl₂ (Promega, Madison, Wis.), 160 μM each deoxynucleotide triphosphate (dNTP) (Statagene, La Jolla, Calif.), 0.4 μM M13 primers (5′-GTT TTC CCA GTC ACG ACG TTG (SEQ ID NO:561) and 5′-GCG GAT AAC AAT TTC ACA CAG GA (SEQ ID NO:562), and 1.25 units Taq polymerase (Promega, Madison, Wis.). The PCR reaction conditions were: 1 cycle at 80° C. (1 min); 1 cycle at 94° C. (2min); 32 cycles at 94° C. (1 min) 57° C. (1 min) 72° C. (2 m); 1 cycle at 72° C. (10 min); and then hold at 4° C. After completion of the PCR reactions the products were purified in a spin-column (Qiagen, Chatsworth, Calif.) and then concentrated in a speed-vac. Aliquots of the PCR products were run on a 1.2% agarose gel containing 0.3 mM ethidium bromide. The gels were digitally imaged using a UVP Bio Doc-It camera (Ultra Violet Laboratory Products, Upland Calif.) and the concentration of each PCR product was determined by comparing the intensity of each band to a standard curve derived from a low DNA mass ladder (Invitrogen Corporation, Carlsbad, Calif.). The PCR products were adjusted to a concentration of 160 ng/μL cDNA template.

Spotting of the macroarrays: The PCR products were loaded into 96 well plates (Fisher Scientific, Pittsburgh, Pa.), denatured with 3 M NaOH, heated to 65° C. for 10 mins, and then immediately quenched on ice. 20× saline sodium citrate (SSC) (3M NaCl, 0.3M sodium citrate, pH 7.0) containing 0.01 mM bromophenol blue was added to the samples to yield a final concentration of 0.3M NaOH, 6×SSC, and 100 ng/μL cDNA template. The PCR products were robotically spotted (Biomek 2000, Beckman Coulter, Fullerton, Calif.) in duplicate onto 11.5 by 7.6 cm neutral nylon membranes (Fisher Scientific) using 100 nL pins. Membranes were UV cross-linked at 1×10⁵ μJoules (UV Stratalinker 1800, Stratagene, La Jolla, Calif.) and stored under vacuum at room temperature until hybridization.

Array controls: Various controls were also spotted onto the membranes, which provided information about cDNA labeling efficiency, blocking at the pre-hybridization step, and non-specific binding. These controls included: 3 Arabidopsis thaliana cDNA clones, Cot-1 repetitive sequences, poly A sequence (SpotReport 3, Stratagene), and a M13 sequence (vector but no cDNA insert). The consistency of the technique was evaluated by spotting on the array multiple cDNA products from the same gene that were amplified in separate PCR reactions.

Sample extraction: Total hepatic messenger ribonucleic acid (mRNA) was extracted using affinity columns (Qiagen, Chatsworth, Calif.) from adult male SHMs treated by aqueous exposure to either 65.14 ng/L of E₂, 109 ng/L EE₂, 100 ng/L DES, 11.81 μg/L pNP, 590.3 ng/L ES or 5.59 μg/L MXC as described previously (Folmar et al., Aquatic Toxicol. 49:77-88, 2000; Hemmer et al., Environ. Toxicol. Chem. 20:336-343, 2001). Three fish were used per treatment group. Criteria for selection of samples from each compound tested were based on previously generated dose response curves (Folmar et al., Aquat. Toxicol. 49:77-88, 2000; Hemmer et al., Environ. Toxicol. Chem. 20:336-343, 2001) and chosen to give similar levels of expression of Vtg mRNA, a well established estrogenic biomarker (Bowman et al., Gen. Comp. Endocrinol. 120:300-313, 2000; Sumpter and Jobling, Environ. Health Perspect. 103:173-178, 1995). By selecting the concentration and length of exposure to yield similar Vtg mRNA expression levels, differing potencies among the chemicals tested was accounted for. Based on this criterion, length of exposure was four days for EE₂ and DES, five days for E₂ and pNP, and thirteen days for MXC. ES treatment levels ranging from 68.8 ng/L to 788.33 ng/L failed to induce Vtg mRNA. A treatment of 590.3 ng/L of ES for these analyses was chosen. This level of ES was slightly below the maximum acceptable toxicant concentration (MATC) derived for ES for SHMs (Hansen and Cripe 1991).

Labeling of RNA and hybridization: Radiolabeled probes were generated by random primer labeling of DNase treated (DNA-free, Ambion, Austin,Tex.) total RNA from male SHM livers with [α-³³P] dATP (Strip-EZ RT, Ambion, Austin, Tex.). The blots were prehybridized with ultraArray hybridization buffer (Ambion, Austin, Tex.) at 64° C. for 3 hours. Following prehybridization, each probe was diluted 20-fold with 10 mM disodium ethylenediaminetetraacetate (EDTA), pH 8.0 to yield 1×10⁶ cpm incorporated ³³P per mL hybridization solution. The diluted probes were heated to 95° C. for 5 mins, quenched on ice for 1 min, and added directly to the prehybridization buffer. The blots were then hybridized overnight at 64° C. Following hybridization, the blots were washed 4×15 minutes each with low (2×SSC, 0.5% SDS) and high (0.5×SSC and 0.5% SDS) stringency washes (Ambion, Austin, Tex.) at 64° C.

Detection and normalization: The membranes were exposed to a phosphor screen (Molecular Dynamics, Piscataway, N.J.) at room temperature for 48 hrs. The blots were quantitatively evaluated using a Typhoon 8600 imaging system (Molecular Dynamics, Piscataway, N.J.). For each cDNA clone, the general background of each membrane was subtracted from the average value of the duplicate spots on the membrane. The values were normalized to the average value of 11 cDNA clones. These genes include ribosomal proteins L8, S9, two unique genes that are similar to ribosomal protein S9, and several clones that do not match any sequences in the National Center for Biotechnology Information (NCBI) database. These genes were chosen to normalize the data because they did not fluctuate appreciably (<1.3 fold) on macro arrays from E₂-treated and control fish and also were shown to be equally expressed in controls and treated fish by DD analysis data. Gene array data was analyzed using linear regression and one-way analysis of variance, with Tukey post-hoc analysis (SigmaStat and SigmaPlot, Jandel, Calif.).

### Results

As a first step toward using array technology, the variability between the macroarrays was determined. To accomplish this, aliquots of identical RNA samples were hybridized onto two separate membranes. A scatter plot correlating the intensity values for each spot on the two membranes was generated. The data points in the graph cluster along a slope of one for all of the spots, including both the low and highly expressed cDNA clones (R²=0.94). Similar R² values ranging from 0.88-0.97 were observed in replicate experiments.

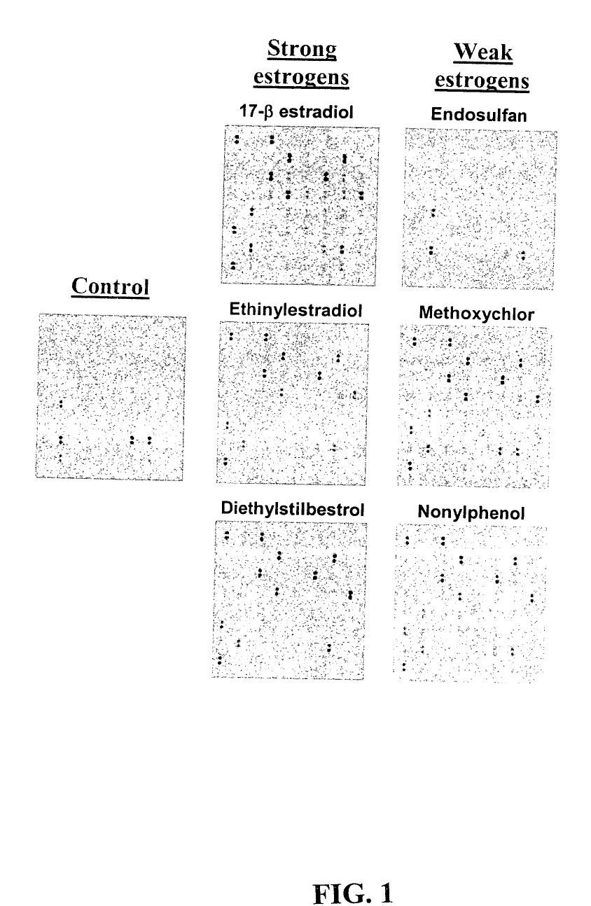

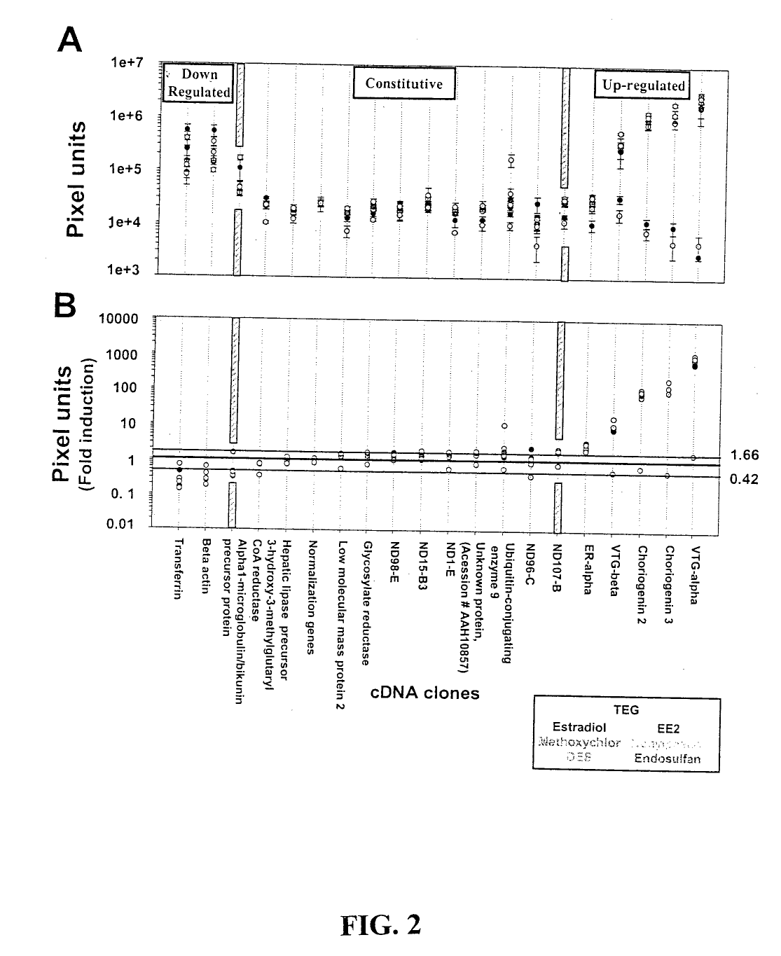

cDNAs corresponding to thirty unique genes were spotted on the macroarrays. These genes were originally isolated by comparing gene expression profiles from control and E₂-treated fish by DD RT-PCR. Hepatic MRNA from exposed fish were radiolabeled and individually hybridized to membranes to determine if fish treated with E₂, EE₂, DES, pNP, MXC, and ES shared similar expression profiles. Three separate fish were used for each treatment. FIG. 1 contains representative membranes from the different treatments and a graphical representation of the data is shown in FIG. 2. FIG. 2A illustrates the mean±SEM intensity values for each of the cDNA clones arranged in order of their expression; FIG. 2B illustrates the mean intensity values for each of the cDNA clones for E₂, EE₂, DES, pNP, MXC or ES divided by the mean intensity values of the respective cDNA clones from the untreated control fish.

Several of the genes that were spotted on the array were found to be up or-down regulated in E₂-treated fish compared to controls. These genes were identified by comparing their intensity values to constitutive genes after correcting for intra-membrane differences based on the intensity values of 11 cDNA clones used to normalize the data. Genes on the macroarray were designated as constitutive if their fold-induction values fell within the range of the mean plus one standard deviation of the highest and lowest values of the 11 clones. Based on this criteria, any cDNA clones in the macroarray experiments above a ˜1.66-fold induction were designated as up-regulated genes respective to control fish, and any cDNA clones that had a value below ˜0.42 were designated as down-regulated.

Of the 30 genes used on the array, 6 genes were found to be up-regulated by E₂ including Vtg α and β, choriogenin 2 and 3, ER α, and coagulation factor XI. Three genes found to be down-regulated by E₂ were transferrin, beta actin, and alpha-1-microglobulin/bikunin precursor protein. The remaining genes did not appear to be differentially regulated by E₂ when compared to controls.

The 9 genes that were up or down-regulated by EE₂, DES, pNP, and MXC exposures showed a similar pattern of expression to the E₂ treatment. Interestingly, ubiquitin-conjugating enzyme 9 was significantly (P<0.05) up-regulated only in the pNP treatments suggesting its regulation is not mediated through the ER. Eight of the nine genes that were found to be up or down-regulated for E₂, EE₂, DES, pNP, and MXC did not fluctuate for ES-treated fish, but instead resembled the pattern observed in control fish. The primary exception was ER α, which appeared to be up-regulated for all of the compounds, including ES. An additional gene, 3-hydroxy-3-methylglutaryl CoA reductase, appeared to be slightly down-regulated in fish treated with ES compared to all of the other treatments and the controls.

To determine if the gene expression profiles on the array could be verified by other techniques that monitor MRNA expression, the expression profiles of several genes on the arrays were compared (Vtg α, choriogenin 2, and transferrin) to their profile by Northern blots and DD RT-PCR. Both Vtg α and choriogenin 2 mRNA levels increase in fish treated with E₂, as measured by Northern blots and DD RT-PCR. Transferrin decreases with E₂ treatment, as measured by Northern blots and DD RT-PCR.

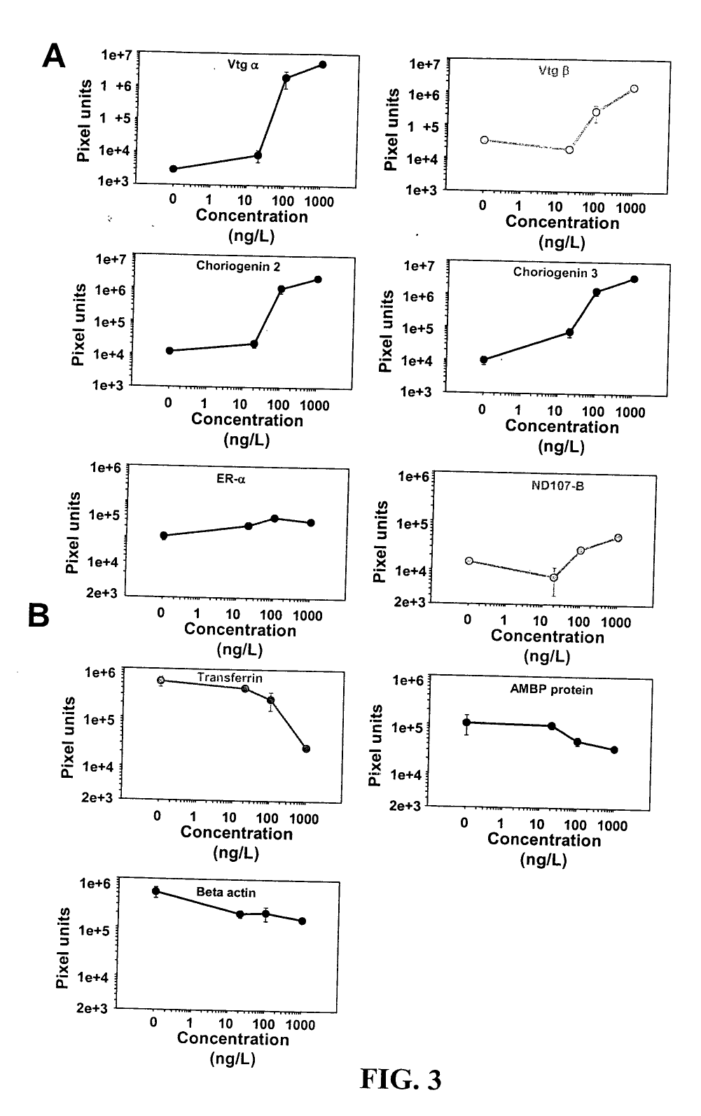

To assess whether the arrays could be used as a quantitative tool to measure the expression of multiple genes at varying concentrations of an estrogenic chemical, male SHMs exposed for 4 days to either 24, 109, or 832 ng/L of EE₂ were examined (Folmar et al., Aquatic. Toxicol. 49:77-88, 2000; Hemmer et al., Environ. Toxicol. Chem. 20:336-343, 2001). FIG. 3 contains graphical illustrations of genes whose expression levels significantly changed more than 2-fold in one or more of the three EE₂ concentrations examined (P<0.05). Vtg α and β, choriogenin 2, choriogenin 3, ER α, and clone ND107-B were found to increase in a concentration dependent manner in the EE₂-exposed fish. Three other genes, transferrin, alpha-1-microglobulin/bikunin precursor protein, and beta actin, appeared to decrease in a dose-dependent manner. These results were consistent with the same genes that were up or down-regulated in the E₂, DES, pNP, and MXC exposed fish (FIG. 2).

### Example 2—Expression Profiling of E₂ Using a SHM Array

A SHM estrogen responsive macroarray was developed to investigate the feasibility of applying array technology in monitoring the environmental distribution of endocrine disrupting compounds that mimic estrogen.

Total hepatic mRNA was extracted from 5 adult male SHMs treated by aqueous exposure to 100 ng/L of E₂ dissolved in triethylene glycol (TEG) for 5 days. Minipreps of 54 cDNA clones derived from DD analysis were PCR amplified using primers specific to the M13 sequence of the cloning vector (pGEMT-Easy, Promega, Madison,Wis.). After the PCR reactions the products were purified in spin-columns (Qiagen, Chatsworth, Calif.) and then concentrated in a speed-vac. The cDNA samples were denatured with NaOH, heated to 65° C. for 10 min, and then immediately quenched on ice. 20×SSC (3M NaCl, 0.3M sodium citrate, pH 7.0) that contained 0.01 mM bromophenol blue was then added to the samples to yield a final concentration of 0.3M NaOH, 6×SSC, and 100 ng/μL cDNA template. The samples were then robotically spotted (Biomek 2000, Beckman Coulter, Fullerton, Calif.) in duplicate onto neutral nylon membranes (Fisher Scientific, Pittsburgh, Pa.) using 100 nL pins. The membranes were UV cross-linked and then stored under vacuum at room temperature until hybridized. Various controls, which provided information about the cDNA labeling efficiency, blocking, and non-specific binding of the arrays, were also spotted onto the membranes. These controls included: 3 Arabidopsis thaliana cDNA clones, Cot-1 repetitive sequences, poly A sequence (SpotReport 3, Stratagene, La Jolla, Calif.), and a M13 sequence (vector but no cDNA insert). Labeling of RNA probes and hybridization of blots was performed as described in Example 1.

The inter-membrane process variability between macroarrays was determined by hybridizing aliquots of identical RNA samples onto two separate membranes. A scatter plot correlating intensity values between the membranes was generated. The data points in the graph clustered along a slope of one (R2 of 0.95, Sigma Stat, Jandel, Calif.), a result which indicates that there is very little variability between membranes.

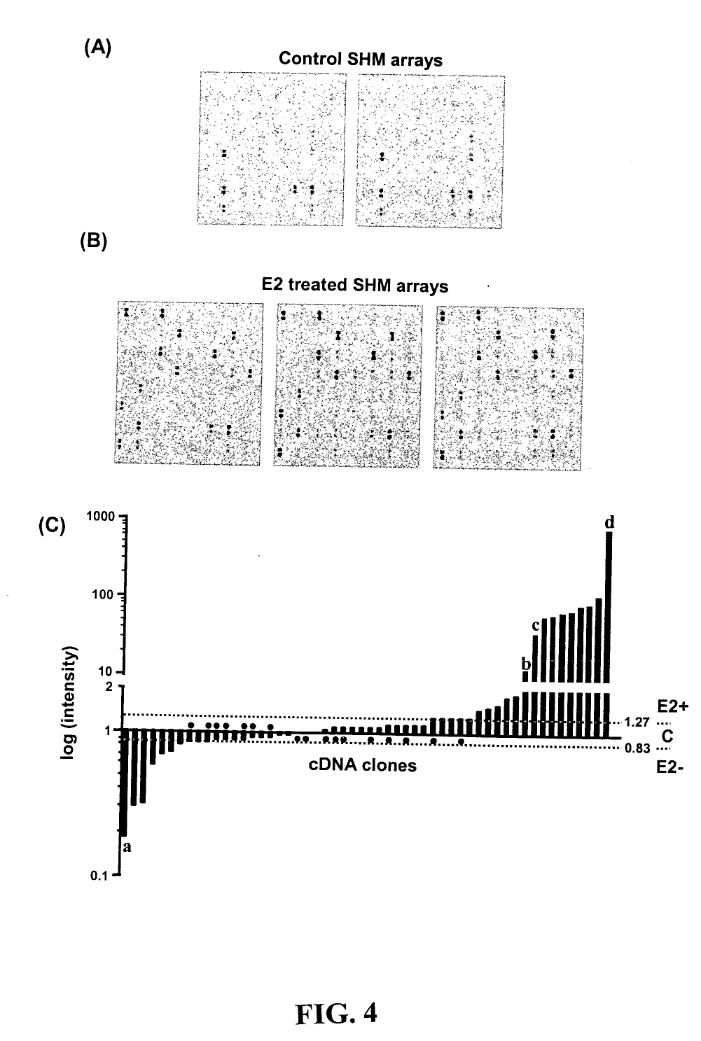

To determine if the gene transcripts found to be up- or down-regulated initially by DD analysis reflect the same induction pattern when spotted onto array membranes, RNA from adult male SHMs aqueously exposed to 100 ng/L of E₂ dissolved in TEG were radiolabeled and hybridized to several membranes. FIGS. 4A and 4B contain blots of control (TEG-treated) and E₂-treated fish, respectively. FIG. 4C is a plot of the mean intensity values. Genes on the macroarray were designated as constitutive genes if their intensity values fell within the range of the highest (1.27) and lowest (0.83) value of the 17 cDNA clones that were used to normalize the data. Based on this criteria, any cDNA clone in the macroarray experiments that had an intensity value above ˜1.27 was designated an E₂ up-regulated gene, and any cDNA clone that had a value below ˜0.83 was designated an E₂ down-regulated gene. Of the 54 cDNA clones that were spotted on the array, 15 genes appeared to be up-regulated by E₂, 32 clones appeared to be constitutive, and 7 genes appeared to be down-regulated by exposure to E₂. All of the highly up-regulated genes, including vitellogenin α and β and the choriogenic protein (ZP2) were also shown to be up-regulated on DD analysis. Interestingly, transferrin, a protein involved in iron transport that was identified to be down-regulated by DD analysis also appears to be down-regulated in response to E₂ on the macroarrays.

### Example 3—Gene Expression Profiles of LMB Exposed to 4-NP and ICI 182,780 Using a LMB Array

Experimental Design and Sample Collection: Adult male LMB were purchased from American Sports Fish Hatchery (Montgomery, Ala.) and maintained in fiberglass tanks as previously described (Larkin et al., Comp. Biochem. Physiol. B. Biochem. Mol. Biol. 133:543-557, 2002; Larkin et al., Marine Environ. Res. 54:395-399, 2002; and Larkin et al., Comparative Biochemistry and Physiology 133:543-557, 2002). Array technology as a tool to monitor exposure of fish to xenoestrogens. Marine Environ. Res., 2002; Bowman et al., Mol. Cell. Endocrinol. 196:67-77, 2002). Each fish was injected IP with either 50 mg/kg 4-NP (Fluka, St. Louis, Mo. # 74430), the combination of 50 mg/kg 4-NP and 1.0 mg/kg ICI 182,780 (Tocris Cookson), or vehicle, which consisted of ethanol and DMSO (Sigma, St. Louis, Mo. #5879). Each dose was dissolved in 1 ml of ethanol and then diluted to the appropriate concentration with DMSO. The fish were euthanized by submersion in a water bath containing 50-100 ppm MS-22 48 hours post injection and sacrificed by a sharp blow to the head followed by cervical transaction. The livers were excised and immediately flash frozen in liquid nitrogen. The frozen tissues were stored at −80° C. until RNA was isolated.

RNA Isolation: Isolation of total RNA from liver tissue was performed with the RNA Stat-60 reagent (Tel-test). Briefly, 30 mg-50 mg of tissue stored in RNA later was homogenized in 0.9 mls STAT 60, choloroform was added, and the mixture was centrifuged at 12,000 g for 15 minutes at 4° C. The extraction process was repeated and the pooled RNA was added to 500 μl isopropanol and allowed to precipitate at −20° C. for at least one hour. Following centrifugation at 12,000 g for 50 minutes, the pellet was washed with 70% ethanol, air dried and resuspended in an appropriate volume (50 μl-120 μl of RNA secure. The samples were treated with RNA secure (Ambion, Austin Tex. #7010) to inactivate contaminating RNases. All isolated RNA was treated with DNase solution (Ambion, Austin, Tex. #1906) following the manufacture's protocol. For all RNA samples, the quantity and quality of total RNA was assessed by spectrophotometric readings at 260 nm and by electrophoresis through a 1% formaldahyde agarose gel stained with ethidium bromide.

Real-Time PCR: Real time PCR was performed using reagents and a 5700 thermocycler purchased from Applied Biosystems (ABI, Foster City, Calif.). The nucleotide sequences of the primers for the ER subtypes and Vtg 1 are as follows: 5′, GACTACGCCTCCGGCTATCAYTATGG (SEQ ID NO:563) AND 5′CATCAGGTAGATCTCAGGGGGYTCNGCNTC (SEQ ID NO:564). Probes and primers for the ER subtypes and Vtg 1 are described in Bowman et al., Ecotoxicology 8:399-416, 1999; and Bowman et al., Mol. Cell Endocrinol. 196:67-77, 2002. Each real time PCR reaction consisted of 0.01-0.2 μg of reverse transcribed total RNA from liver tissue, 1× universal Taqman master mix (ABI, Foster City, Calif.), and primers and probes in a 25 μl reaction. To generate a standard curve, varying amounts of plasmid containing the specific cDNA inserts for each gene were used as template in the PCR reactions. For each gene, a 6 point standard encompassing a 1×10⁶ fold range of approximately 25-2.5×10⁶ copies of cDNA was constructed. Each sample was run in duplicate and normalized 18s rRNA, also obtained by real-time PCR. Both the intra-assay and inter-assay variability never exceeded 10%. The final data is graphed as the mean and standard error of the relative copies of each ER or Vtg MRNA per μg of total RNA. Statistical differences between the treatments were determined by one way analysis of variance with Dunnets post-hoc analysis.

Amplification of cDNA to be spotted on the macro arrays: The macroarrays were prepared and printed as previously described (Larkin et al., Marine Environ. Res. 54:395-399, 2002). Briefly, the 132 LMB clones were PCR amplified using primers specific to the M13 sequence of the cloning vector (pGEMT-Easy, Promega, Madison,Wis.). After completion of the PCR reactions the products were purified using MultiScreen PCR plates (Millipore, Bedford, Mass.), concentrated, denatured with NaOH, heated to 65° C. for 10 min, and then immediately quenched on ice. 20×SSC (3M NaCl, 0.3M sodium citrate, pH 7.0) containing 0.01 mM bromophenol blue was added to the samples to yield a final concentration of 0.3M NaOH, 6×SSC, and 100 ng/μL cDNA template. The PCR products were robotically spotted (Biomek 2000, Beckman Coulter, Fullerton, Calif.) in duplicate onto neutral nylon membranes (Fisher Scientific, Pittsburgh, Pa.) using 100 nL pins. Membranes were UV cross-linked and stored under vacuum at room temperature until hybridization.

Labeling of RNA and hybridization was performed as described in Example 1. The membranes were exposed to a phosphor screen (Molecular Dynamics, Piscataway, N.J.) at room temperature for 48 hours. The blots were quantitatively evaluated using a Typhoon 8600 imaging system (Molecular Dynamics, Piscataway, N.J.). For each cDNA clone, the general background of each membrane was subtracted from the average value of the duplicate spots on the membrane. The values were normalized to the average value of 12 cDNA clones specific to ribosomal genes, which included S2, S3, S8, S15, S16, S27, L4, L5, L8, L13, L21, and L28. Ribosomal genes were chosen to normalize the data because they do not appear to fluctuate appreciably (<1.3 fold) in response to estrogenic compounds. Genes were not included for analysis that had values less than the background value for two out of the three replicates and/or fluctuated more then two fold when aliquots of the same RNA were hybridized to blots printed at the beginning, middle, and the end of the array printing process.

Measurement of ER and Vtg 1 mRNA by real-time PCR: Real-time PCR is a sensitive assay that can be used to quantitate expression levels of genes. Using this technology, assays were designed to quantitate the expression of 4 genes, estrogen receptors alpha, beta, and gamma, and Vtg 1 in LMB following exposure to 4-NP and 4-NP/ICI 182,780. Using primers and probes specific to each gene it was possible to differentiate between the ER isotypes with no cross reactivity. Exposure of LMB to a single IP injection of 4-NP (50 mg/kg) significantly increased ER α by 80 fold (p <0.05) after 48 hours when compared to controls. During the same time frame, the levels of both ER β and ER γ decreased approximately 1.3-fold and 2.6-fold respectively, however these changes were not statistically significant from controls. When the LMB were exposed to a combination of 4-NP (50 mg/kg) and the anti-estrogen ICI 182,780 (1.0 mg/kg), the levels of ER a increased only 4-fold over controls (p<0.08), suggesting that the anti-estrogen had interfered with the activation process. As with the 4-NP treatment, the expression of ER β and γ decreased (1.9-fold) but the values did not differ significantly from controls.

Since the Vtg gene is an E₂-responsive gene that is under transcriptional control by ERs in the liver, the expression levels of Vtg 1 were also determined by real-time PCR. Exposure to 4-NP increased message levels by approximately 40-fold over controls (p<0.05), however, this induction was not repressed by the addition of ICI 182,780.

LMB gene array analysis: In order to further characterize the effects of 4-NP alone or in conjunction with ICI 182,780 on hepatic gene regulation in LMB, the expression of 132 genes was examined, many of which are estrogen responsive, by gene arrays. Total hepatic RNA isolated from control and exposed fish was radiolabeled and hybridized to the membranes. Of the 132 genes on the array, only genes that changed by at least 3 standard deviations from the mean of the 12 ribosomal genes that were used to normalize the data are included. These include several that are up or down-regulated by more than 2-fold, a conservative cutoff generally used for array interpretation. The mean and standard error for each gene for control and treated LMB was determined. The fold induction of each gene over controls for both the NP and NP/ICI 182,780 treatments was determined.

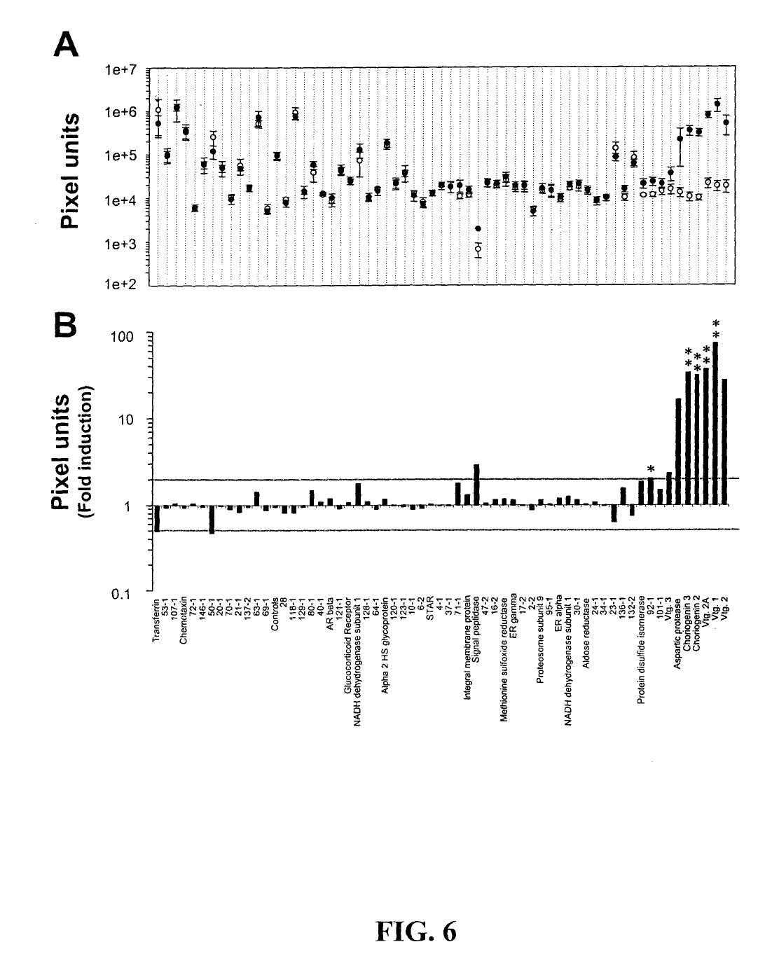

In the 4-NP-treated fish (FIG. 6), 9 genes were up-regulated 2-fold or greater including 4 Vtgs, choriogenin 2, choriogenin 3, aspartic protease, signal peptidase, and one unidentified clone designated 92-1. Two genes were found to be down-regulated by 4-NP including transferrin and clone 50-1. In the case of the mixture of 4-NP and ICI 182,780, some genes that were up-regulated by 4-NP treatment alone were reduced, but not all. In fact, the expression levels of 4 Vtgs, 2 choriogenins, and transferrin were not affected at all; instead they appear to be expressed to the same levels as with the 4-NP alone. Vtg 1, 2, 2a, and 3 were induced approximately 74, 28, 37, and 2-fold over controls respectively. The levels of both choriogenins increased to values approximately 35-fold over controls while aspartic protease was induced 16 fold over controls.

Genes which were reduced by the mixture and that exhibited at least a 2-fold change in expression included aspartic protease, protein disulfide isomerase, integral membrane protein, methionine sulfoxide reductase, ER γ, glucocorticoid receptor, aldose reductase, ER β, FK506 binding protein, and 21 unidentified clones. All of these genes except for clone 53-1 were down regulated by the addition of ICI 182,780 to the 4-NP.

### Example 4—Gene Expression Analysis of LMB Exposed to E₂ and p,p′-DDE Using a LMB Array Materials and Methods

Amplification of cDNA to be spotted on the macro arrays: The 132 clones of LMB genes in pGEM-T Easy plasmids were PCR amplified in a 300 μL reaction containing 1×PCR Buffer A (Promega, Madison,Wis.), 2 mM MgCl₂ (Promega, Madison, Wis.), 160 μM each dNTP (Statagene, La Jolla, Calif.), 0.4 μM M13 primers (5′-GTT TTC CCA GTC ACG ACG TTG (SEQ ID NO:?) and 5′-GCG GAT AAC AAT TTC ACA CAG GA (SEQ ID NO:?)), and 1.25 units Taq polyrnerase (Promega, Madison, Wis.). The PCR reaction conditions were 1 cycle at 80° C. (1 min), 1 cycle at 94° C. (2min), 32 cycles at 94° C. (1 min), 57° C. (1 min), and 72° C. (2 min), 1 cycle at 72° C. (10), and then hold at 4° C. After completion of the PCR reactions the products were purified using MultiScreen PCR plates (Millipore, Bedford, Mass.) and then concentrated in a speed-vac. Aliquots of the PCR products were run on a 1.2% agarose gel containing 0.3 mM ethidium bromide. The gels were digitally imaged using a UVP Bio Doc-It camera (Ultra violet Laboratory Products, Upland Calif.) and the concentration of each PCR product was determined by comparing the intensity of the gel band to a standard curve derived from a low DNA mass ladder (Invitrogen Corporation, Carlsbad, Calif.). The PCR products were adjusted to a concentration of 160 ng/μL cDNA template.

Spotting of the gene arrays and various controls used are described in Example 1. Chemicals, Treatment, and Preparation of the hepatic samples: E₂ (# E-8875) and p, p′-DDE (#12,389-7) were obtained from Sigma-Aldrich Corporation (St Louis, Mo.); 4-NP #74430, 85% para isomer) was obtained from Fluka (Milwaukee, Wis.).

Adult (˜1.5 year old) LMB weighing 300±71 grams were obtained from American Sports Fish Hatchery (Montgomery, Ala.). Fish were acclimated for a minimum of one month in an aerated holding tank prior to treatment. The fish were exposed to ambient light and fed Purina Aquamax 5D05 fish chow (St. Louis, Mo.). Groups of fish received a single IP dose of E₂ (2.5 mg/kg), 4-NP (50 mg/kg), or p, p′-DDE (100 mg/kg). E₂ and 4-NP were dissolved in 1 mL of 100% ethanol and then diluted to the appropriate concentration with DMSO (Sigma, St. Louis, Mo. # 5879), whereas p, p′-DDE was dissolved directly in DMSO. Control fish received an IP injection of the ethanol/DMSO or DMSO diluent without any chemical. During the experimental period the fish were not fed.

The fish were euthanized 48 hours after the IP injection by addition of 50-100 parts per million (ppm) of tricaine methanesulfonate (MS-222) to the water followed by a sharp blow to the head and cervical transection. The livers were excised from the fish and immediately flash frozen with liquid nitrogen. Total RNA was extracted from the tissue samples using RNeasy affinity columns (Qiagen, Chatsworth, Calif.).

Labeling of RNA and hybridization was performed as described in Example 1. Detection and normalization was performed as described in Example 3. Transcript data were analyzed using linear regression and student t-tests (SigmaStat and SigmaPlot, Jandel, Calif.).

### Results

Gene array technology has enabled researchers to analyze hundreds to thousands of genes on a single array. As a first step toward using array technology, the inter membrane variability between the gene arrays was determined. To accomplish this, aliquots of identical RNA samples were hybridized onto two separate membranes. A scatter plot correlating the intensity values for the cDNA clones between the two arrays was generated. The data points in the graph cluster along a slope of one starting with the low to the high expressed cDNA clones (R2 of 0.98). Similar results were observed in a replicate experiment.

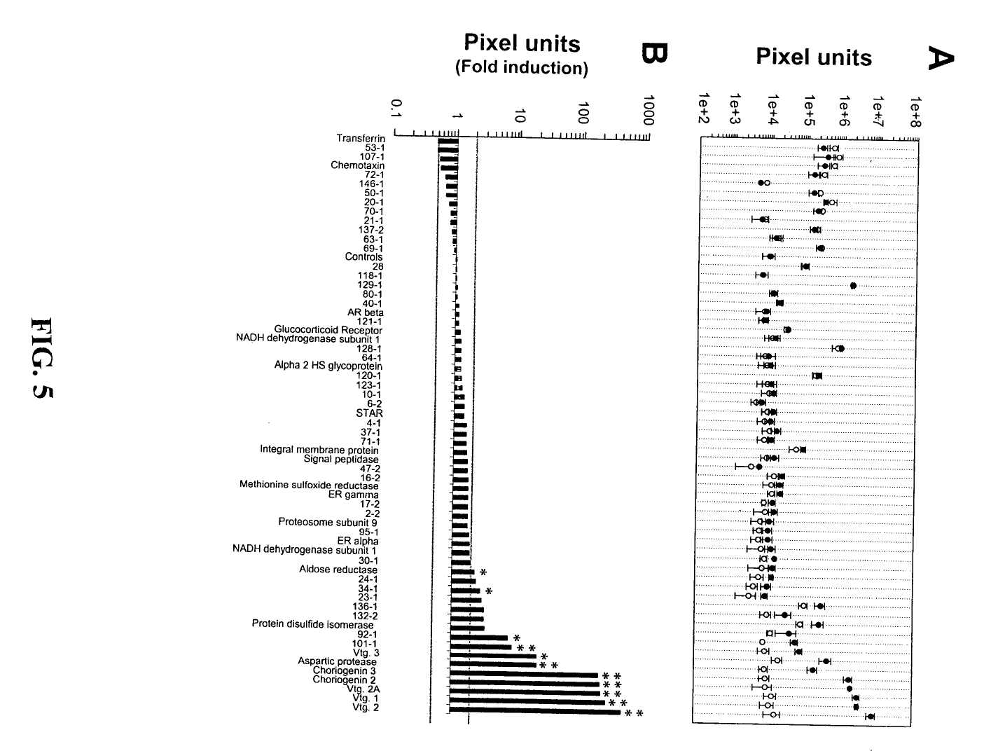

In order to determine the specific expression profile of 132 unique genes in LMB exposed to E₂, or to the contaminants 4-NP and p, p′-DDE, hepatic total RNAs from exposed fish were radiolabeled and individually hybridized to separate membranes. Three separate fish were used for each treatment. A graphical representation of this data is shown in FIG. 5. FIG. 5A illustrates the mean±SEM intensity values for each of the cDNA clones arranged in order of their expression; FIG. 5B illustrates the mean intensity values for each of the cDNA clones for E₂ divided by the mean intensity values of the respective cDNA clones from control fish. Only genes from any of the treatments (E₂, NP or DDE) that were 3 standard deviations from the mean (0.98±0.41) of the 12 r-protein genes that were used as constitutive controls are shown. While there are a number of genes whose expression levels meet this criterion, only genes that exhibit a two-fold or greater change in expression were considered to be differentially regulated. A two-fold cutoff is commonly used by researchers to demarcate up or-down regulated genes for array experiments (Nagahama, Y. Int. J. Dev. Biol. 38:217-229, 1994; Lin and Peter, Comp. Biochem. Physiol. B. Biochem. Mol. Biol. 129:543-550, 2001). Of the 132 genes used on the array, 16 genes were up-regulated 2-fold or greater by E₂ including four Vtg genes, choriogenin 2, choriogenin 3, aspartic protease, protein disulfide isomerase, aldose reductase, and 7 unidentified clones designated 23-1, 24-1, 34-1, 92-1, 101-1, 132-2, and 136-1. Two genes were down-regulated two-fold or more by E₂ including transferrin and a clone designated 53-1.

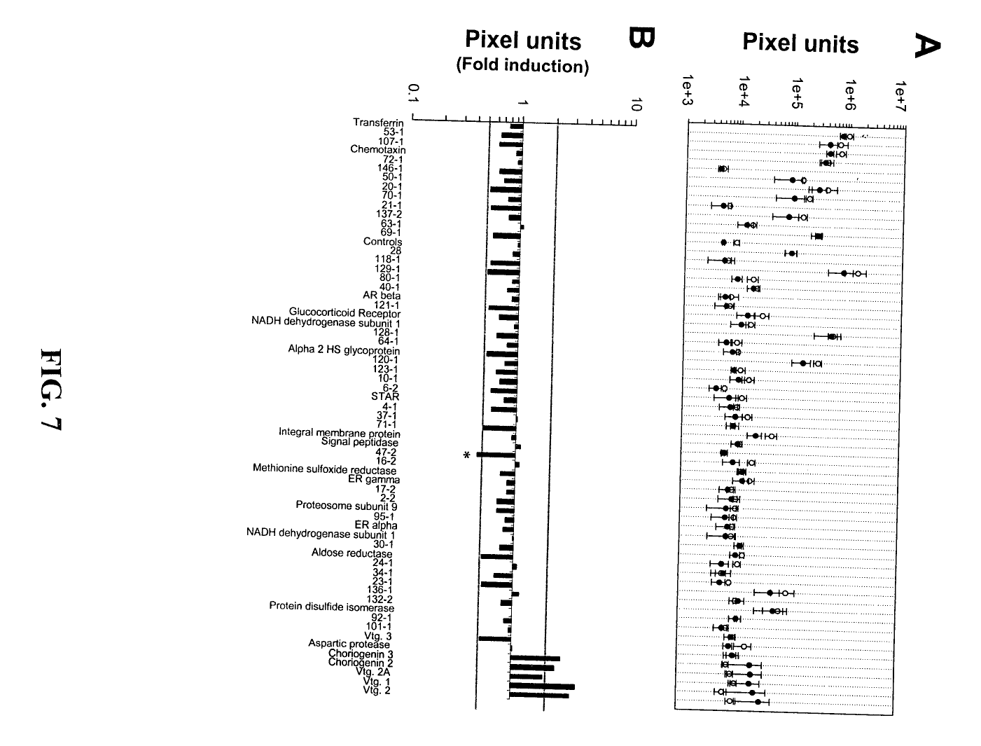

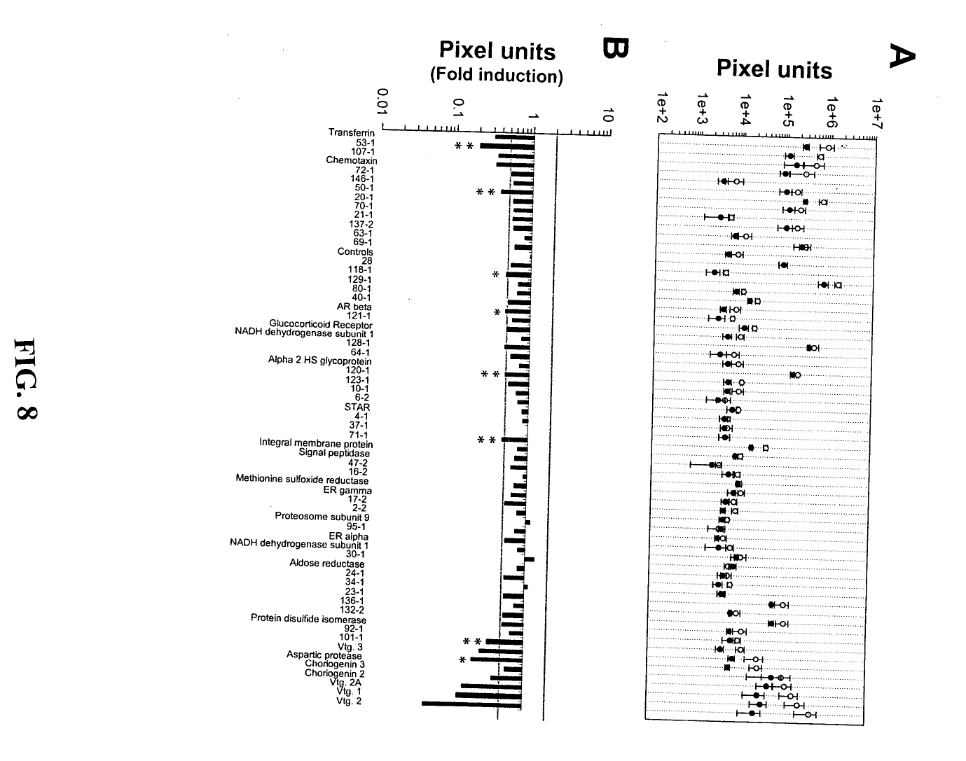

Since the mode of action of p, p′-DDE has not been extensively characterized, the influences of this compound on the expression profiles of the 132 genes arrayed in both male and female fish was examined. In male fish (FIG. 7), four genes were up-regulated by p, p′-DDE including Vtg 1, Vtg 2, choriogenin 2, and choriogenin 3, whereas one gene, clone 47-2 was down-regulated. In female fish (FIG. 8) injected with p, p′-DDE, no genes were identified as up-regulated; however, 17 genes were down-regulated two-fold or greater. These included the four Vtg 's, aspartic protease, transferrin, chemotaxin, choriogenin 2, androgen receptor, and 8 unidentified clones designated 50-1, 53-1, 71-1, 101-1, 107-1, 118-1,120-1, and 128-1.

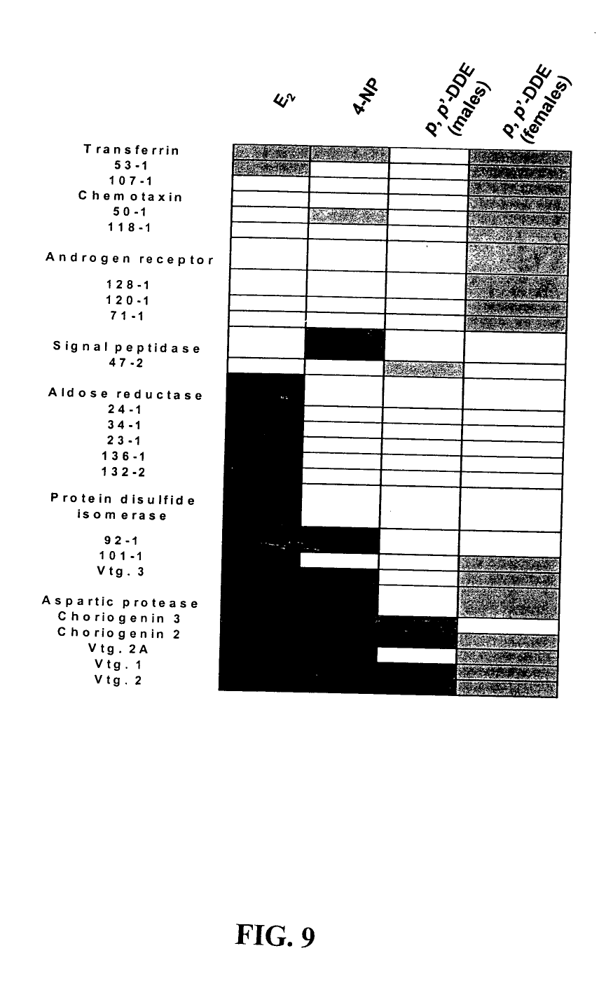

Summaries of the genes whose expression increased or decreases more than 2-fold for each exposure are depicted in FIG. 9. Light shading indicates down-regulated genes while dark shading indicates up-regulated genes.

### Example 5—Altered Gene Expression in Liver of LMB Exposed To Androgens Methods

Suppressive subtractive hybridization: Juvenile LMB were treated with a single 50 μl intraperitoneal (IP) injection of either a 2 μM solution of dihydrotestosterone (DHT) or progesterone in DMSO (˜2.5 nmol/g BWT). Fish were euthanized four days later and their livers were removed. Hepatic polyA+ RNA was isolated from these samples and subtractive hybridizations (Clontech, Palo Alto, Calif.) were performed in one direction using DMSO as the driver. The subtracted gene pools were then cloned into pGEM T-Easy (Promega, Madison, Wis.) and sequenced. Clones were identified using tBlastx at the National Center for Biotechnology Information (NCBI).

Gene arrays: cDNAs obtained from SSH were arrayed as previously described (Larkin et al., Marine Environ. Res. 54:395-399, 2002) and then hybridized with 33P-labeled single-stranded cDNAs isolated from adult male LMB treated with 62.5 μg/g DHT or 20 μg/g 11-ketotestosterone (11-KT) or vehicle (DMSO) (n=5 per treatment). For each cDNA clone, the general background of each membrane was subtracted from the average value of the duplicate spots on the membrane. The values were then normalized to the average value of seven cDNA clones specific to ribosomal genes and the fold change calculated by dividing the mean of each treatment by the mean of the control. Those genes which changed by 2-fold or more were graphed. Significant differences (p<0.05) were determined by ANOVA and secondary testing was done by using Tukey's LSD.

### Results

The results are shown in Table 1. Genes that were the most elevated include Vtg 2, spermidine-spermine N¹-acetyltransferase (SSAT), and ZPCs 1 and 4, while the LDL receptor, RXR interacting protein, and Vtg receptor were the most decreased. While the patterns of regulation appeared similar for both androgens, some specific differences did occur. For instance, aspartic protease and glutathione peroxidase III were up- and down-regulated, respectively, by DHT alone. Conversely, a fish homolog to pituitary tumor transforming protein (PTTP) and cystatin were up- and down-regulated, respectively by 11-KT alone. One gene that was up-regulated by both androgens was sSAT. This gene was shown to be unaffected by estradiol in the pig (Green et al., Biol. Reprod. 59:1251-1258, 1998).

TABLE 1
Genes Up/Down Regulated By 11-KT and DHT

| | | | TREATED | |
|---|---|---|---|---|
| | | | WITH 20 | CHANGE |
| | | | MG/KG | BY |
| LOC | Gene ID | E-score | DHT | 11KT |
| D14 | VTG PRECURSOR | 1.33E−39 | up | up |
| C13 | SSAT | 0 | up | up |
| H7 | EST SEASONAL 64 | 6.3 | up | up |
| F2 | 97-8 | | up | NC |
| E12 | EST SEASONAL 88 | 4 | up | NC |
| O11 | RIKEN 1110001M01 | 2.61E−05 | up | up |
| B14 | EST SEASONAL 62 | 9.04 | up | up |
| B13 | SOLUTE CARRIER | 2.29E−37 | up | up |
| F12 | EST SEASONAL 56 | 1.7 | up | NC |
| G13 | RHAMNOSE BINDING | 1.00E−43 | up | NC |
| | LECTIN | | | |
| J13 | TFIIIA | 1.20E−30 | up | NC |
| H14 | ZPC1 | 0 | up | up |
| D13 | EST SEASONAL 9 | 7.58 | up | up |
| I14 | ZPC4 | 2.58E−25 | up | up |
| B8 | EST SEASONAL 12 | | up | NC |
| I7 | ASP PROT | | up | NC |
| C14 | UNNAMED PROTEIN | 1.48E−36 | up | NC |
| E3 | ATPASE 6 | 3.17E−18 | up | NC |
| F9 | ATPASE SUBUNIT 6 | 5.13E−24 | up | NC |
| M11 | RETINOL | 9.07E−38 | up | up |
| | DEHYDROGENASE | | | |
| K7 | ATP SYNTHASE | 1.35E−08 | up | NC |
| K13 | ESTP4_H07 | 2.16 | up | up |
| O13 | EST SEASONAL F21 | 0.82 | down | down |
| F5 | ESTDHT60 | 6.92 | down | NC |
| L2 | ALPHA 1 ANTI- | 2.80E−27 | down | NC |
| | TRYPSIN | | | |
| D12 | RIKEN 2700038C09 | 1.50E−04 | down | NC |
| M4 | EST SEASONAL 55 | 1.32 | down | down |
| B2 | 53-1 | | down | NC |
| C2 | 68-1 | | down | NC |
| M5 | IGF-I | 7.50E−03 | down | NC |
| D6 | ESTP4_E06 | 6.7 | down | NC |
| G9 | ESTP4_C04 | 1.78 | down | NC |
| I5 | HAPTOGLOBIN | 5.19E−28 | down | NC |
| K2 | ALDOLASE B | 0 | down | NC |
| C3 | APOLIPOPROTEIN E | 1.13E−26 | down | NC |
| K12 | EST SEASONAL 72 | 6.57 | down | down |
| M2 | ALPHA TUBULIN | 2.93E−40 | down | down |
| G5 | GLUTATHIONE | 0 | down | NC |
| | PEROXIDASE III | | | |
| K5 | ESTP4_E01 | 3.43E+00 | down | NC |
| L1 | 24-1 | | down | down |
| L14 | ER GAMMA 5′ 2F | | down | down |
| J5 | HEPCIDIN | 2.49E−23 | down | NC |
| L3 | COMPLEMENT C3 | 3.20E−04 | down | down |
| M12 | TFIID (change to | 1.88E−07 | down | down |
| | liver regeration | | | |
| | related protein) | | | |
| K11 | EST SEASONAL 51 | 0.39 | down | down |
| K1 | GP3 11C | | down | down |
| L11 | RXR INTERACTING | 1.36E−09 | down | down |
| | PROT | | | |
| M14 | VTG RC | | down | down |
| J10 | LDL RC | 1.62E−32 | down | down |
| L9 | EST SEASONAL 90 | | down | NC |
| N8 | EST P4_D08 | | up | NC |
| O10 | PTTP | 3.12E−22 | up | NC |
| D4 | EST DHT64 | 0.322 | up | NC |
| E14 | warm water acclim | 3.18E−12 | up | up |
| L8 | EST P4_06 | | up | NC |
| H10 | EST SEASONAL 42 | 0.011 | down | NC |
| B9 | EST SEASONAL F17 | | down | NC |
| O6 | EST SEASONAL 11 | 0.36 | down | NC |
| O3 | CYSTATIN | 8.39E−05 | down | NC |

### Example 6—LMB And SHM Genes Up/down-regulated in Response to Estrogenic Agents

TABLE II
LMB Gene Regulation

| | | Differentially |
|---|---|---|
| LMB# | Gene ID | expressed by |
| LMB_COMP FACTOR | Putative complement | |
| Bf/C2 | factor Bf/C2 | |
| LMB_ABMP | ABMP precursor | |
| LMB GLUT-PEROX III | Glutathione | Dn-reg DHT |
| | peroxidase III | |
| LMB_Srnp D1 | Small ribonucleoprotein | |
| | D1 polypeptide (16 kD) | |
| LMB_RIBO L6 | Ribosomal protein L6 | |
| LMB_MYOSIN LIGHT | myosin regulatory | |
| | light chain | |
| LMB_ZPC1 | ZPC1 | up-reg DHT; |
| | | 11-KT |
| LMB_CYTO-C OX 1 | Cytochrome c oxidase | |
| | subunit I | |
| LMB_LECTIN STL2 | Rhamnose binding | up-reg DHT |
| | lectin STL2 | |
| LMB_EMAP2 | Echinoderm microtubule | |
| | associated protein | |
| | like 2 | |
| LMB_ALDOLASE-B | Aldolase b | |
| LMB_RIBO L7A | 60S ribosomal | |
| | protein L7A | |
| LMB_PROTHROMBIN | Prothrombin precursor | |
| LMB_SSAT | SSAT | up-reg DHT; |
| | | 11-KT |
| LMB_COMPLEMENT- | Complement C3 precursor | Dn-reg DHT; |
| C3 | | 11-KT |
| LMB_RIBO L7 | Ribosomal protein L7 | |
| LMB H-ATPASE- | H+-ATPase subunit, | |
| SUBUNIT | oligomycin sensitivity | |
| | conferring protein | |
| LMB_RIBO L23A | Ribosomal protein L23a | |
| LMB_ALPHA-TUBULIN | alpha tubulin | Dn-reg 11-kt; |
| | | DHT |
| LMB_RIBO-Sa | 40S ribosomal | |
| | protein Sa | |
| LMB_VTG | Vitellogenin prcursor | |
| LMB NASCENT-POLYPEP | Nascent polypeptide- | |
| | associated complex, | |
| | alpha polypeptide | |
| LMB_ApoH | Apoliporotein H | |
| LMB_TBT-BP | TBT-binding protein | |
| LMB_SOL-CAR- | solute carrier family | up-reg DHT; |
| 25A#5 | 25 alpha member 5 | 11-KT |
| LMB_UNNAMED- | Unnamed protein | |
| PROTEIN | product | |
| LMB_FIB-B-SUBUNIT | Fibrinogen B subunit | |
| LMB CIS-RETIN | cis-retinol | up-reg DHT |
| DEHYDRO | dehydrogenase | |
| LMB_SENES-ASSOC | Putative senscence- | |
| PROTEIN | associated protein | |
| LMB_LDL RC | LDL receptor | Dn-reg DHT; |
| | | 11-KT |
| LMB_ABC-TRANS | ABC transporter | |
| LMB_CATHEPSIN B | Cathepsin B | |
| LMB_SERPIN-CP9 | Serpin CP9 | |
| LMB_TFIIIA | Transcription factor | |
| | IIIA (TFIIIA) | |
| LMB_ANTITHROMBIN | Antithrombin III | |
| III | | |
| LMB_RIKEN | RIKEN cDNA | |
| 1810056020 | 1810056020 | |
| LMB_WEE-I | Wee I tyrosine | |
| | kinase | |
| LMB_HAPTOGLOBIN | Haptoglobin | Dn-reg DHT |
| LMB_APOA-I | APOPLIPOPROTEIN A-I | |
| LMB_ALPHA-1 | alpha-1 antitrypsin | Dn-reg DHT |
| ANTITRYPSIN | homolog precursor | |
| LMB_APOE | Apolipoprotein E | |
| LMB_ZPC4 | ZPC4 | up-reg DHT; |
| | | 11-KT |
| LMB_LECTIN 9 | C-type lectin | |
| | superfamily 9 | |
| LMB_ATPASE 6 | ATPase subunit 6 | up-reg DHT |
| LMB_ITI | inter-alpha-trypsin | |
| | inhibitor “ITI” | |
| LMB_EIF-3#7 | Eukaryotic translation | |
| | initiation factor 3 | |
| | subunit 7 | |
| LMB_HEPCIDIN | Hepcidin precursor | dn-reg DHT |
| LMB_PTTP | Pituitary tumor | |
| | transforming protein | |
| LMB_TOXIN-1 | Toxin-1 | |
| LMB_COAG FACTOR | Coagulation factor | |
| VII | VII | |
| LMB_CDC42-2 | cdc 42 isoform 2 | |
| LMB_WARM-WATER | Warm water acclimation- | up-reg 11-KT |
| ACC PROTEIN | related protein | |
| LMB_CYTO-C OX II | Cytochrome c oxidase | |
| | subunit II | |
| LMB_L10A | 60S ribosomal | |
| | protein L10A | |
| LMB_KALLIKREIN | Kallikrein | |
| LMB_DANIO EST | Danio EST | |
| 3818635 | IMAGE: 3818635 | |
| LMB_ALPHA-2- | alpha-2- | |
| MACROGLOB-1 | macroglobulin-1 | |
| LMB_HAPTOGLOB | Haptoglobin- | |
| RELATED PROT | related protein | |
| LMB_FILAMEN-B | Filamen B | |
| LMB_UBIQUITIN | ubiquitin | |
| LMB_RXR INTERACT | Retinoid X receptor | Dn-reg 11-KT; |
| PROT | interacting protein | DHT |
| LMB_MITOCHON- | ATP synthase alpha | up-reg DHT |
| ATP-SYNTHASE | chain mitochondrial | |
| | precursor | |
| LMB_TATA BOX BP | TATA-box binding | |
| | protein | |
| LMB_DIFF-REG | Differentially | |
| TROUT PROT-1 | regulated trout | |
| | protein 1 | |
| LMB LIVER-REGEN- | liver regeneration | |
| REL PROT | related protein | |
| LMB_SERPIN-2B | Serpin 2b | |
| LMB_APO-A1 | Apolipoprotein | |
| | A-I-1 precursor | |
| LMB_M-PHASE | M-phase | |
| PROT 6 | phosphoprotein 6 | |
| LMB_PROSTAGLAND- | Prostaglandin D | |
| D-SYNTHASE | synthase-like protein | |
| | (lipocalin type) | |
| LMB_LYRIC | LYRIC | |
| LMB CYSTATIN-PREC | Cystatin precursor | Dn-reg 11-KT |
| LMB_RIKEN 2700038 | RIKEN cDNA 2700038 | |
| LMB_DIAZEPAM- | Membrane associated | |
| BINDING INHIB | diazepam-binding | |
| | inhibitor | |
| LMB_IGF-I | IGF-I | |
| LMB_ESTP4_D11 | ESTP4_D11 | |
| LMB_ESTDHT_6 | ESTDHT_6 | |
| LMB_ESTDHT_7 | ESTDHT_7 | |
| LMB_ESTDHT_13 | ESTDHT_13 | |
| LMB_ESTDHT_50 | ESTDHT_50 | |
| LMB_ESTDHT_51 | ESTDHT_51 | |
| LMB_ESTDHT_53 | ESTDHT_53 | |
| LMB_ESTDHT_60 | ESTDHT_60 | |
| LMB_ESTDHT_62 | ESTDHT_62 | up-reg DHT; |
| | | 11-KT |
| LMB_ESTDHT_68 | ESTDHT_68 | |
| LMB_ESTDHT_69 | ESTDHT_69 | |
| LMB_ESTP4_A02 | ESTP4_A02 | |
| LMB_ESTP4_B03 | ESTP4_B03 | |
| LMB_ESTP4_B04 | ESTP4_B04 | |
| LMB_ESTP4_B07 | ESTP4_B07 | |
| LMB_ESTP4_B08 | ESTP4_B08 | |
| LMB_ESTP4_B09 | ESTP4_B09 | |
| LMB_ESTP4_C03 | ESTP4_C03 | |
| LMB_ESTP4_C04 | ESTP4_C04 | |
| LMB_ESTP4_C06 | ESTP4_C06 | |
| LMB_ESTP4_D04 | ESTP4_D04 | |
| LMB_ESTP4_D08 | ESTP4_D08 | |
| LMB_ESTP4_D10 | ESTP4_D10 | |
| LMB_ESTP4_E01 | ESTP4_E01 | |
| LMB_ESTP4_E03 | ESTP4_E03 | |
| LMB_ESTP4_E06 | ESTP4_E06 | |
| LMB_ESTP4_E08 | ESTP4_E08 | |
| LMB_ESTP4_E12 | ESTP4_E12 | |
| LMB_ESTP4_F06 | ESTP4_F06 | |
| LMB_ESTP4_G06 | ESTP4_G06 | |
| LMB_ESTP4_G11 | ESTP4_G11 | |
| LMB_ESTP4_H02 | ESTP4_H02 | |
| LMB_ESTP4_H04 | ESTP4_H04 | |
| LMB_ESTP4_H05 | ESTP4_H05 | |
| LMB_ESTP4_H07 | ESTP4_H07 | |
| LMB_ESTP4_H08 | ESTP4_H08 | |
| LMB_EST- | EST-SEASONAL_02 | |
| SEASONAL_02 | | |
| LMB_EST- | EST-SEASONAL_03 | |
| SEASONAL_03 | | |
| LMB_EST- | EST-SEASONAL_04 | |
| SEASONAL_04 | | |
| LMB_EST- | EST-SEASONAL_06 | |
| SEASONAL_06 | | |
| LMB_EST- | EST-SEASONAL_09 | up-reg DHT; |
| SEASONAL_09 | | 11-KT |
| LMB_EST- | EST-SEASONAL_11 | dn-reg 11-KT |
| SEASONAL_11 | | |
| LMB_EST- | EST-SEASONAL_12 | up-reg DHT |
| SEASONAL_12 | | |
| LMB_EST- | EST-SEASONAL-14 | |
| SEASONAL-14 | | |
| LMB_EST- | EST-SEASONAL_16 | |
| SEASONAL_16 | | |
| LMB_EST- | EST-SEASONAL_17 | |
| SEASONAL_17 | | |
| LMB_EST- | EST-SEASONAL_22 | |
| SEASONAL_22 | | |
| LMB_EST- | EST-SEASONAL_51 | dn 11-KT; |
| SEASONAL_51 | | DHT |
| LMB_EST- | EST-SEASONAL_52 | |
| SEASONAL_52 | | |
| LMB_EST- | EST-SEASONAL_54 | |
| SEASONAL_54 | | |
| LMB EST- | EST-SEASONAL_55 | Dn-reg 11-KT; |
| SEASONAL_55 | | DHT |
| LMB EST- | EST-SEASONAL_56 | up-reg DHT; |
| SEASONAL_56 | | |
| LMB EST-- | EST-SEASONAL_58 | |
| SEASONAL_58 | | |
| LMB EST-- | EST-SEASONAL_59 | |
| SEASONAL_59 | | |
| LMB EST-- | EST-SEASONAL_61 | |
| SEASONAL_61 | | |
| LMB EST-- | EST-SEASONAL_62 | |
| SEASONAL_62 | | |
| LMB EST-- | EST-SEASONAL_64 | up-reg DHT; |
| SEASONAL_64 | | 11KT |
| LMB EST-- | EST-SEASONAL_68 | |
| SEASONAL_68 | | |
| LMB EST-- | EST-SEASONAL_70 | |
| SEASONAL_70 | | |
| LMB EST-- | EST-SEASONAL_71 | |
| SEASONAL_71 | | |
| LMB EST-- | EST-SEASONAL_72 | Dn-reg DHT; |
| SEASONAL_72 | | 11KT |
| LMB EST-- | EST-SEASONAL_75 | |
| SEASONAL_75 | | |
| LMB EST-- | EST-SEASONAL_77 | |
| SEASONAL_77 | | |
| LMB EST-- | EST-SEASONAL_85 | |
| SEASONAL_85 | | |
| LMB EST-- | EST-SEASONAL_88 | up-reg DHT; |
| SEASONAL_88 | | |
| LMB EST-- | EST-SEASONAL_90 | dn-reg DHT |
| SEASONAL_90 | | |
| LMB EST-- | EST-SEASONAL_92 | |
| SEASONAL_92 | | |
| LMB EST-- | EST-SEASONAL_97 | |
| SEASONAL_97 | | |
| LMB EST-- | EST-SEASONAL_F11 | |
| SEASONAL_F11 | | |
| LMB EST-- | EST-SEASONAL_F17 | Dn-reg 11-KT |
| SEASONAL_F17 | | |
| LMB EST-- | EST-SEASONAL_F21 | Dn-reg DHT |
| SEASONAL_F21 | | |
| LMB_ER-ALPHA | ESTROGEN RECEPTOR | up-reg E2; |
| | ALPHA | NP |
| LMB_ER-BETA | ESTROGEN RECEPTOR | |
| | BETA | |
| LMB_ER-GAMMA | ESTROGEN RECEPTOR | Dn-reg 11-KT; |
| | GAMMA | up-reg E2 |
| LMB_STAR | STAR PROTEIN | up-reg cAMP; |
| | | dn-reg b- |
| | | sitosterol |
| LMB_SF1 | SF1 PROTEIN | |
| | FRAGMENT | |
| LMB_ESTP4-E01 | LMB_ESTP4-E01 | down by DHT |
| LMB_ESTDHT64 | LMB_ESTDHT64 | up by 11KT |
| LMB LIV-REGER- | LMB_LIV- | down by DHT |
| PROT | REGER-PROT | |
| LMB RIKEN | LMB_RIKEN | up by 11KT; |
| 1110001M01 | 1110001M01 | DHT |
| LMB EST- | LMB_EST-SEASONALf17 | down by 11KT |
| SEASONALf17 | | |
| LMB1-3 | unknown | |
| LMB2-2 | unknown | |
| LMB3-1 | unknown | |
| LMB4-1 | unknown | |
| LMB5 | vitellogenin-2A | Up reg E2; |
| | | NP; Dn-reg |
| | | DDE(F) |
| LMB6-1 | AMBP protein precursor | |
| LMB7-1 | Unknown | |
| LMB8-2 | Unknown | |
| LMB9-1 | Unknown | |
| LMB10-1 | Unknown | |
| LMB11-2 | Unknown | |
| LMB12-1 | Zebrafish | |
| | Oligosaccharyl | |
| | transferase integral | |
| | membrane protein | |
| LMB13-2 | Unknown | |
| LMB14-1 | Unknown | |
| LMB15-1 | NADH dehydrogenase | |
| | subunit 1 | |
| LMB16-2 | unknown | |
| LMB17-2 | Mitochondrial control | |
| | region | |
| LMB18-3 | unknown | |
| LMB19-1 | Insulin like growth | |
| | factor | |
| LMB20-1 | unknown | |
| LMB21-1 | unknown | |
| LMB22-1 | unknown | |
| LMB23-1 | unknown | Up-reg E2 |
| LMB24-1 | Unknown | Up-reg E2, |
| | | dn-reg DHT; |
| | | 11-KT |
| LMB25-1 | Ribosomal porotein | |
| | S8 | |
| LMB26-1 | Transferrin | |
| LMB27-1 | unknown | |
| LMB28-2 | unknown | |
| LMB29-2 | unknown | |
| LMB30-1 | unknown | |
| LMB31 | choriogenin | |
| LMB32-1 | G-box binding factor | |
| | (bacteria) | |
| LMB33-1 | unknown | |
| LMB34-1 | unknown | up-reg E2 |
| LMB35-1 | unknown | |
| LMB36-1 | hypothetical protein | |
| LMB37-1 | unknown | |
| LMB38-1 | 40S ribosomal | |
| | protein S2 | |
| LMB39-1 | unknown | |
| LMB40-1 | alport syndrome | |
| | chrom region gene | |
| LMB41-1 | ribosomal protein L8 | |
| LMB42-1 | Gamma fibrinogen | |
| LMB43-1 | FK506 binding protein, | |
| | immunophillin | |
| LMB44-1 | Dynein heavy chain | |
| LMB45-1 | vitellogenin A | |
| LMB46-1 | unknown | |
| LMB47-2 | unknown | Down-reg |
| | | DDE(M) |
| LMB48-1 | elongation factor | |
| | 1 beta | |
| LMB49-1 | 40S ribosomal | |
| | protein S15 | |
| LMB50-1 | unknown | Down reg NP; |
| | | DDE(F) |
| LMB51-1 | unknown | |
| LMB52-1 | unknown | |
| LMB53-1 | unknown | Down-reg. E2; |
| | | DDE (F); DHT |
| LMB54-2 | L4 ribosomal | |
| LMB55-1 | L4 ribosomal | |
| LMB56-1 | 40S ribosomal | |
| LMB57 | ADP, ATP translocase | |
| LMB58-1 | ribosomal L21 | |
| LMB59-1 | Unknown | |
| LMB60-1 | unknown, AK010552 | |
| LMB61-1 | unknown | |
| LMB63-1 | unknown | |
| LMB64-1 | unknown | |
| LMB65-1 | unknown | |
| LMB66-1 | unknown | |
| LMB67-1 | signal peptidase, | Up-reg NP |
| | endopeptidase | |
| LMB68-1 | hypothetical protein | Dn-reg DHT |
| LMB69-2 | unknown | |
| LMB70-2 | NADH dehydrogenase | |
| | subunit 1 | |
| LMB71-1 | unknown | down-reg |
| | | DDE (F) |
| LMB72-1 | unknown | |
| LMB73-1 | NADH dehydrogenase | |
| | subunit 1 | |
| LMB74-3 | unknown | |
| LMB75-1 | 40S ribosomal | |
| LMB76-2 | unknown | |
| LMB77-1 | unknown | |
| LMB78-1 | unknown | |
| LMB79-2 | unknown | |
| LMB80-1 | unknown | |
| LMB81-1 | unknown | |
| LMB82-1 | unknown | |
| LMB83 | vitellogenin-2 | up-reg E2; |
| | | NP; DDE(M); |
| | | dn-reg DDE(F) |
| LMB84-1 | STAR | |
| LMB85-1 | CYP1A | |
| LMB86-1 | ribosomal protein | |
| | L28 | |
| LMB87 | vitellogenin-1 | up-reg E2; |
| | | NP; DDE(M); |
| | | dn-reg DDE(F) |
| LMB88-1 | unknown | |
| LMB89-1 | glucocorticoid | |
| | receptor | |
| LMB90-1 | unknown | |
| LMB91-1 | unknown | |
| LMB92-1 | unknown | up-reg E2; |
| | | NP |
| LMB93-1 | estrogen receptor | |
| | gamma | |
| LMB94-1 | transferrin | Down-reg. |
| | | E2; NP; |
| | | DDE in F |
| LMB95-1 | CAP-rich Zinc finger | |
| | protein | |
| LMB96-1 | unknown | |
| LMB97 | choriogenin-3 | Up-reg E2; |
| | | NP; DDE(M); |
| | | DHT |
| LMB98-1 | estrogen receptor | |
| | beta | |
| LMB99-1 | estrogen receptor | |
| | alpha | |
| LMB100-1 | ribosomal protein L5 | |
| LMB101-1 | unknown | Up-reg E2, Dn |
| | | for DDE(F) |
| LMB102-1 | chemotaxin | down-reg |
| | | DDE (F) |
| LMB103-1 | proteosome subunit 9 | |
| LMB104-3 | 60S ribosomal protein | |
| | L13 | |
| LMB105-1 | unknown | |
| LMB107-1 | unknown | down-reg |
| | | DDE (F) |
| LMB108-1 | choriogenin-2 | Up-reg E2; NP; |
| | | DDE(M); Dn- |
| | | reg DDE (F) |
| LMB109-1 | 40S ribosomal | |
| | protein S3A | |
| LMB110-1 | Methionine sulfoxide | |
| | reductase | |
| LMB112-1 | cathepsin (Aspartic | up-reg E; NP: |
| | protease) | DHT; Dn-DDE (F) |
| LMB116-1 | aldose reductase | up-reg E2 |
| LMB118-1 | apolipoprotein | down-reg |
| | precursor | DDE (F) |
| LMB120-1 | hypothetical protein | donw-reg |
| | | DDE (F) |
| LMB121-1 | TBT binding protein | |
| LMB122-1 | alpha2-HS | |
| | glycoprotein | |
| LMB123-1 | Urocanase | |
| LMB128-1 | unknown | down-reg |
| | | DDE (F) |
| LMB129-1 | unknown | |
| LMB130-1 | secreted phosphoprotein | |
| | precursor | |
| LMB132-1 | integrin beta | up-reg E2 |
| LMB133-1 | unknown | |
| LMB134-3 | unknown | |
| LMB135 | protein disulfide | up-reg E2 |
| | isomerase | |
| LMB136-1 | protein disulfide | up-reg E2 |
| | isomerase like | |
| LMB137-2 | unknown | |
| LMB138-1 | unknown | |
| LMB139-1 | apolipoprotein C2 | |
| LMB140-1 | unknown | |
| LMB141 | vitellogenin-3 | up reg E2; NP, |
| | | dn DDE (F) |
| LMB142-1 | hypothetical protein | |
| | (FLJ10530) | |
| LMB144-1 | vitellogenin like | |
| LMB150 | androgen receptor | Dn-reg DDE(F) |
| LMB151 | vitellogenin receptor | Dn-reg |
| | | DHt-11-KT |

TABLE III
SHM Gene Regulation

| Clone ID | Identity | E value | Regulation |
|---|---|---|---|
| SHM IK 7A | 40 S ribosomal protein | 2.00E−39 | |
| | (Ictalurus punctatus) | | |
| SHM IK 24E | similar to ribosomal protein | 4.00E−34 | |
| | L37a, cytosolic | | |
| SHM IK 25C | ribosomal protein L5 | 5 E−05 | |
| SHM IK 5D | 60 S ribosomal protein L8 | 5.00E−26 | |
| SHM IKIGF-1 | IGF I | | |
| SHM IKIGF-2 | IGF 2 | | |
| Female Test (SSH) | | | |
| ndSHM-FT1-A03 | sertotransferrin precursor | 1.00E−99 | |
| | (O. Latipes) | | |
| ndSHM-FT1-A09 | putative transmembrane | 7.88E−08 | up-reg- E2 |
| | 4 superfamily member protein | | |
| ndSHM-FT1-A10 | unknown | | up-reg-E2 |
| ndSHM-FT1-A11 | phospholipid hydroperoxide | 5.61E−44 | dn-reg E2 |
| | glutathione peroxidase | | |
| ndSHM-FT1-A12 | sertotransferrin precursor | 3.90E−35 | dn-reg E2 |
| | (O. Latipes) | | |
| ndSHM-FT1-B03 | Unknown | | |
| ndSHM-FT1-B07 | Similar to aldehyde dehydrogenase | 0 | |
| | 7 family, member A1 | | |
| ndSHM-FT1-B10 | cytochrome b | 0 | |
| | [Orestias silustani] | | |
| ndSHM-FT1-C01 | Similar to high mobility group | 0 | |
| | box 1 [Danio rerio] | | |
| ndSHM-FT1-C03 | perforin 1 (pore forming | 2.00E−19 | up reg E2 |
| | protein) human,, | | |
| ndSHM-FT1-C04 | Prostaglandin D Synthase | 1.01E−05 | dn-reg E2 |
| | [Xenopus laevis] | | |
| ndSHM-FT1-C09 | endoplasmic reticulum lumenal | 0 | dn-reg E2 |
| | L-amino acid oxidase | | |
| ndSHM-FT1-D06 | Unknown | | up-reg E2 |
| ndSHM-FT1-D10 | unknown | | up-reg E2 |
| ndSHM-FT1-D12 | unknown | | dn-reg E2 |
| ndSHM-FT1-E01 | probable complement regulatory | 2.79E−09 | dn-reg E2 |
| | plasma protein SB1 - | | |
| ndSHM-FT1-E02 | Cytochrome C oxidase subunit II | 2.00E−62 | |
| ndSHM-FT1-E08 | unknown | | up-reg E2 |
| ndSHM-FT1-E09 | unknown | | up-reg E2 |
| ndSHM-FT1-E12 | Similar to chitinase, (D. rerio) | 1.00E−83 | dn-reg E2 |
| ndSHM-FT1-F01 | leucine-rich alpha-2-glycoprotein | 3.29E−13 | |
| | [Homo sapiens] | | |
| ndSHM-FT1-F06 | complement component C3 | 0 | dn-reg E2 |
| | [Paralichthys olivaceus] | | |
| ndSHM-FT1-F09 | solute carrier family 27 (fatty | 6.13E−19 | |
| | acid transporter), member | | |
| ndSHM-FT1-F10 | beta hemoglobin A | 1.00E−42 | dn-reg E2 |
| | [Seriola quinqueradiata] | | |
| ndSHM-FT1-F11 | unknown | | dn-reg E2 |
| ndSHM-FT1-F12 | | | up reg E2 |
| ndSHM-FT1-G02 | unknown | | |
| ndSHM-FT1-G04 | Unknown | 2.15847 | up-reg E2 |
| ndSHM-FT1-G08 | endoplasmic reticulum lumenal | 0 | |
| | L-amino acid oxidase | | |
| ndSHM-FT1-H02 | FUGRU complement component | 3.00E−16 | |
| | C9 precursor | | |
| ndSHM-FT1-H03 | 35 kDa serum lectin | 1.85E−35 | |
| | [Xenopus laevis] | | |
| ndSHM-FT1-H04 | Similar to chitinase, acidid | 4.00E−83 | dn-reg E2 |
| | (D. rerio) | | |
| ndSHM-FT1-H06 | SPI-2 serine protease inhibitor | 1.97E−09 | |
| | (AA 1-407) [Rattus no | | |
| ndSHM-FT1-H07 | unknown | | up-reg E2 |
| ndSHM-FT1-H10 | Unknown | | |
| ndSHM-FT1-H11 | unknown | | up-reg E2 |
| ndSHM-FT1-H12 | beta hemoglobin A | 1.40E−45 | up-reg E2 |
| ndSHM-MC1-A02 | Liver basic fatty acid bp | 2.00E−43 | dn-reg E2 |
| ndSHM-MC1-A03 | Polyadenylate-binding protein 1 | 0 | |
| ndSHM-MC1-A04 | unknown | | up-reg E2 |
| ndSHM-MC1-A05 | beta galactosidase/ubiquitin | 3.00E−44 | |
| | fusion protein | | |
| ndSHM-MC1-A07 | Orla C3 (O. latipes) | 9.00E−38 | |
| ndSHM-MC1-A09 | alpha-2-macroglobulin 2 | 2.00E−06 | dn-reg E2 |
| | (C. carpio) | | |
| ndSHM-MC1-A11 | alpha-1-antitrypsin | 1.27E−11 | dn-reg E2 |
| | [Sphenodon punctatus] | | |
| ndSHM-MC1-B01 | unknown | | dn-reg E2 |
| ndSHM-MC1-B03 | cytochrome c oxidase, subunit Va | 0 | |
| ndSHM-MC1-B04 | KIAA0018 protein [Homo sapiens] | 9.29E−35 | up-reg E2 |
| ndSHM-MC1-B05 | Unknown | | up-reg E2 |
| ndSHM-MC1-B08 | complement component C5-1 | 5.61E−30 | |
| | [Cyprinus carpio] | | |
| ndSHM-MC1-B10 | Serotransferrin precursor >gi\| | 2.00E−39 | |
| | 1814091\|dbj\|BAA10901.1\| | | |
| ndSHM-MC1-B11 | fibrinogen, B beta polypeptide | 2.80E−45 | dn-reg E2 |
| ndSHM-MC1-C02 | Similar to fibrinogen, gamma | 4.06E−41 | up-reg-field |
| | polypeptide [Danio rerio] | | |
| | | | dn-reg E2 |
| ndSHM-MC1-C04 | 4-hydroxy-phenylpyruvate- | 0 | |
| | dioxygenase | | |
| ndSHM-MC1-C05 | unknown | | up-reg E2 |
| ndSHM-MC1-C08 | serine proteinase inhibitor | 8.08E−39 | dn-reg E2 |
| | CP9 - common carp | | |
| ndSHM-MC1-C10 | prothrombin precursor | 0 | |
| | [Takifugu rubripes] | | |
| ndSHM-MC1-D01 | | | |
| ndSHM-MC1-D02 | ATPase, H+ transporting, | 1.47E−25 | |
| | lysosomal, | | |
| ndSHM-MC1-D03 | fatty acid binding protein 2, | 2.00E−58 | up-reg-field |
| | hepatic (Japanese seapearch) | | |
| ndSHM-MC1-D04 | Proteasome Regulatory Particle, | 0 | |
| | ATPase-like | | |
| ndSHM-MC1-D06 | expressed sequence AL022852 | 1.63E−21 | up-reg E2 |
| | [Mus musculus] | | |
| ndSHM-MC1-D10 | Scavenger receptor with C/type | 5.00E−14 | dn-reg E2 |
| | lectine type I (Human) | | |
| ndSHM-MC1-E01 | similar to monocarboxylate | 2.73E−17 | |
| | transporter 6 | | |
| ndSHM-MC1-E05 | elastase 4 precursor | 0 | up-reg field |
| | [Paralichthys | | |
| ndSHM-MC1-E06 | Unknown | | |
| ndSHM-MC1-E08 | pre alpha inhibitor heavy | 3.00E−14 | dn-reg E2 |
| | chain 3 rat | | |
| ndSHM-MC1-E10 | Unknown | | up-reg E2 |
| ndSHM-MC1-E12 | unknown | | up-reg E2 |
| ndSHM-MC1-F01 | similar to charged amino acid | 1.83E−11 | up-reg E2 |
| | rich leucine zipper factor-1 | | |
| ndSHM-MC1-F02 | chemotaxis (O. mykiss) | 2.00E−60 | |
| ndSHM-MC1-F03 | dendritic cell protein | 0 | |
| | [Homo sapiens] | | |
| ndSHM-MC1-F06 | Chain A, Alcohol Dehydrogenase | 0 | |
| ndSHM-MC1-F11 | 17-beta-hydroxysteroid | 0 | up-reg E2 |
| | dehydrogenase type IV | | |
| ndSHM-MC1-F12 | interferon induced protein 2 | 2.49E−07 | up-reg E2 |
| | [Ictalurus punctatus] | | |
| ndSHM-MC1-G01 | Alcohol dehydrogenase >gi\| | 0 | up-reg E2 |
| | 482344\| | | |
| ndSHM-MC1-G02 | 14 kDa apolipoprotein | 1.45E−16 | dn-reg E2 |
| | [Anguilla japonica] | | |
| ndSHM-MC1-G03 | serine (or cysteine) | 1.14E−29 | dn-reg E2 |
| | proteinase inhibitor, | | |
| | clade F | | |
| ndSHM-MC1-G04 | ribosomal protein XL1a - | 0 | |
| | African clawed frog | | |
| ndSHM-MC1-G05 | microfibrillar-associated | 3.27E−13 | |
| | protein 4 | | |
| ndSHM-MC1-G07 | apolipoprotein E | 4.21E−37 | |
| | [Scophthalmus maximus] | | |
| ndSHM-MC1-G11 | aldehyde reductase AFAR2 | 3.34E−36 | up-reg E2 |
| | subunit [Rattus norvegicus] | | |
| ndSHM-MC1-G12 | Similar to RIKEN cDNA | 4.21E−12 | |
| | 1300018K11 gene [Homo sapiens] | | |
| ndSHM-MC1-H02 | unknown | | |
| ndSHM-MC1-H03 | complement factor B/C2B | 8.00E−28 | |
| | (O. mykiss) | | |
| ndSHM-MC1-H04 | similar to ribosomal protein | 1.07E−23 | up-reg field |
| | S25, cytosolic [validated] - | | |
| ndSHM-MC1-H06 | unnamed protein product | 0.000421546 | up-reg E2 |
| | [Homo sapiens] | | |
| ndSHM-MC1-H08 | peroxisomal proliferator- | 2.00E−08 | |
| | activated receptor beta1 | | |
| | [Salmo salar] | | |
| ndSHM-MC1-H09 | Ligand-gated ionic channel | 1.93158 | up-reg E2 |
| | family member | | |
| ndSHM-MC1-H10 | unknown | 3.75692 | up-reg E2 |
| ndSHM-MC1-H12 | Similar to sperm associated | 4.36E−18 | |
| | antigen 7 [Homo sapiens] | | |
| Male Test SSH | | | |
| ndSHM-MT1-A02 | chicken fatty acid binding | 3.00E−52 | up-reg E2 |
| | protein | | |
| ndSHM-MT1-A03 | warm temperature acclimation | 6.00E−40 | |
| | related 65 kDa protein | | |
| | (O. latipes) | | |
| ndSHM-MT1-A05 | Transducin beta/like 2 protein | e−107 | up-reg E2 |
| ndSHM-MT1-B09 | putative mitochonrial inner | 1.00E−34 | up-reg E2 |
| | membrane protease subunit | | |
| | (Human) | | |
| ndSHM-MT1-C05 | unknown | | up-reg E2 |
| ndSHM-MT1-C08 | vitellogenin I | 6.89E−43 | up-reg E2 |
| | [Cyprinodon variegatus] | | |
| ndSHM-MT1-D04 | WS beta-transducin repeats | 1.87E−05 | |
| | protein [Homo sapiens] | | |
| ndSHM-MT1-D05 | mesau serum amyloid A/3 | 5.00E−25 | up-reg E2 |
| | protein precursor | | |
| ndSHM-MT1-D07 | vitellogenin (Sillago japonica) | 8.00E−78 | up-reg E2 |
| ndSHM-MT1-E02 | 40 S ribosomal protein S3 | E−105 | |
| ndSHM-MT1-E03 | Similar to transducin (beta)- | 0 | up-reg E2 |
| | like 2 [Xenopus laevis] | | |
| ndSHM-MT1-E05 | Predicted CDS, seven TM | 3.00782 | up-reg E2 |
| | Receptor S | | |
| ndSHM-MT1-F11 | Similar to transducin (beta)- | 1.42E−16 | |
| | like 2 [Xenopus laevis] | | |
| ndSHM-MT1-G03 | 60S ribosomal protein | 2.40E−38 | |
| | L10a > g | | |
| ndSHM-MT1-H05 | Similar to transducin (beta)- | 0 | |
| | like 2 [Xenopus laevis] | | |
| METHOXYCHLOR- | | | |
| CONTROL SSH | | | |
| ndSHM-MXCc1- | Protein involved in recombination | 0.0928205 | dn-reg E2 |
| A04 | repair, homologous to S. pombe | | |
| | rad18. | | |
| ndSHM-MXCc1- | no hit | | |
| A09 | | | |
| ndSHM-MXCc1- | KIAA0096 gene product is | 5.62E−12 | up-reg E2 |
| A10 | related to a protein kinase. | | |
| ndSHM-MXCc1- | alpha s HS glycogrotein | 1.00E−47 | |
| A11 | (Platichthys flesus) | | |
| ndSHM-MXCc1- | dodecenoyl-Coenzyme A | 3.82E−37 | up-reg E2 |
| B02 | delta isomerase | | |
| ndSHM-MXCc1- | cytochrome P450 3A56 | 0 | up-reg field |
| B03 | [Fundulus heteroclitus] | | |
| ndSHM-MXCc1- | kallistatin | 4.93156 | up-reg E2 |
| B04 | [Rattus norvegicus] | | |
| ndSHM-MXCc1- | Fibrinogen beta chain precursor | 5.78E−24 | dn-reg E2 |
| B06 | [Contains: Fibrinopeptide B] | | |
| ndSHM-MXCc1- | Apolipoprotein A/I precursor | 5.00E−25 | dn-reg E2 |
| B07 | (sparus aurata) | | |
| ndSHM-MXCc1- | Similar to retinol dehydro- | 4.16E−32 | |
| B08 | genase type III [Danio rerio] | | |
| ndSHM-MXCc1- | Beta-2-glycoprotein I precursor | 1.92E−11 | |
| C04 | (Apolipoprotein H) ( | | |
| ndSHM-MXCc1- | tyrosine kinase [Gallus gallus] | 1.31312 | |
| C06 | | | |
| ndSHM-MXCc1- | ceruloplasmin [Danio rerio] | 0 | up-reg |
| C11 | | | field |
| ndSHM-MXCc1- | vitellogenin I precursor | 4.00E−51 | up-reg E2 |
| D03 | (Mummichog) | | |
| ndSHM-MXCc1- | hypothetical protein | 0.826071 | dn-reg E2 |
| D04 | [Ferroplasma acidarmanus] | | |
| ndSHM-MXCc1- | cytochrome c oxidase subunit I | 8.28E−35 | dn-reg E2 |
| D05 | [Engraulis japonicus] | | |
| ndSHM-MXCc1- | no hit | | up-reg E2 |
| D08 | | | |
| ndSHM-MXCc1- | Immunoglobulin domain- | 0.991091 | up-reg E2 |
| D10 | containing protein family | | |
| ndSHM-MXCc1- | hypothetical protein | 8.1324 | |
| D12 | [Plasmodium falciparum 3D7] | | |
| ndSHM-MXCc1- | hypothetical protein | 0.61028 | |
| E01 | [Magnetospirillum magnetotacticum] | | |
| ndSHM-MXCc1- | unknown protein | | up-reg E2 |
| E09 | | | |
| ndSHM-MXCc1- | sorting nexin 11 [Homo sapiens] | 0 | |
| E11 | | | |
| ndSHM-MXCc1- | vitellogenin B (M. aeglefinus) | 6.00E−16 | up-reg E2 |
| F01 | | | |
| ndSHM-MXCc1- | warm-temperature-acclimation- | 6.25E−25 | dn-reg E2 |
| F03 | related-protein- [Oryzias latipes] | | |
| ndSHM-MXCc1- | UDP-glucose pyrophosphorylase | 0 | |
| F07 | [Gallus gallus] | | |
| ndSHM-MXCc1- | interferon-related developmental | 6.68E−39 | up-reg E2 |
| F10 | regulator 1 [Mus musculus] | | |
| ndSHM-MXCc1- | unknown protein | 0.202018 | up-reg E2 |
| G02 | | | |
| ndSHM-MXCc1- | thyroid hormone receptor | 0 | up-reg E2 |
| G03 | interactor 12; | | |
| ndSHM-MXCc1- | Putative ribosomal protein L21 | 0 | |
| G04 | | | |
| ndSHM-MXCc1- | putative delata 6-desaturase | 0 | up-reg E2 |
| G12 | [Oncorhynchus masou] | | |
| ndSHM-MXCc1- | complement control protein | 1.72E−23 | |
| H05 | factor I-A [Cyprinus carpio] | | |
| ndSHM-MXCc1- | ATP synthase 6 | 3.00E−23 | |
| H09 | | | |
| METHOXYCHLOR | | | |
| TEST SSH | | | |
| ndSHM-MXCt1- | rat liver regeneration related | 1.00E−48 | |
| B05 | protein | | |
| ndSHM-MXCt1- | BH2041 ˜unknown conserved | 6.52356 | up-reg E2 |
| B08 | protein [Bacillus halodurans] | | |
| ndSHM-MXCt1- | lysophospholipase (Rat) | 1.00E−36 | up-reg E2 |
| C02 | | | |
| ndSHM-MXCt1- | Unknown protein for MGC:63946 | 3.00E−29 | |
| C11 | (D. rerio) | | |
| ndSHM-MXCt1- | unknown | | up-reg E2 |
| D09 | | | |
| ndSHM-MXCt1- | unknown | | up-reg E2 |
| E04 | | | |
| ndSHM-MXCt1- | CG4198-PA [Drosophila | 0.385852 | |
| E06 | melanogaster] | | |
| ndSHM-MXCt1- | PROBABLE IRON OXIDASE | 3.15365 | |
| E09 | PRECURSOR OXIDOREDUCTASE | | |
| | PROTEIN | | |
| ndSHM-MXCt1- | Vitellogenin I precursor | 0 | up-reg E2 |
| E12 | (VTG I) [Contains: Lipovitellin 1 ( | | |
| ndSHM-MXCt1- | Unknown | | up-reg E2 |
| F11 | | | |
| ndSHM-MXCt1- | Unknown | | |
| G03 | | | |
| ndSHM-MXCt1- | miro2 pending protein | 4.00E−60 | |
| H03 | | | |
| ndSHM-MXCt1- | Group XIII secretory | 6.05E−40 | up-reg E2 |
| H09 | phospholipase A2 precursor | | |
| NONYLPHENOL | | | |
| CONTROL SSH | | | |
| ndSHM-NPc1-A12 | unknown | | |
| ndSHM-NPc1-B01 | NADH subunit 1 | 2.80E−45 | up-reg field |
| | [Cyprinodon variegatus] | | |
| ndSHM-NPc1-B08 | Chain A, Complex Of The | 1.20E−15 | up-reg field |
| | Catalytic Portion Of Human | | |
| ndSHM-NPc1-B09 | calreticulin [Danio | 0 | |
| | rerio] >gi\|6470259\|gb\| | | |
| ndSHM-NPc1-C04 | hypothetical protein APE0566 - | 0.667761 | |
| ndSHM-NPc1-C06 | Vitellogenin II precursor (VTG II) | 0 | up-reg E2 |
| | [Fundulus heteroclitus] | | |
| ndSHM-NPc1-C11 | translation elongation factor | 7.14E−10 | |
| | 1-alpha [Stylonychia mytilus] | | |
| ndSHM-NPc1-E01 | Vitellogenin I | 1.20E−33 | up-reg E2 |
| | [Cyprinodon variegatus] | | |
| ndSHM-NPc1-E06 | Unknown | | |
| ndSHM-NPc1-E11 | Unknown | | up-reg E2 |
| ndSHM-NPc1-F01 | ubiquitin A-52 residue ribosomal | 2.00E−37 | |
| | protein [Homo sapiens] | | |
| ndSHM-NPc1-F05 | Vitellogenin A | 0.000293022 | up-reg E2 |
| | [Melanogrammus aeglefinus] | | |
| ndSHM-NPc1-F06 | Unknown | | up-reg E2 |
| ndSHM-NPc1-F07 | LFA-3 (delta TM) [Ovis sp.] | 0.0763225 | up-reg E2 |
| ndSHM-NPc1-F08 | CG32659-PA | 0.0316684 | |
| | [Drosophila melanogaster] | | |
| ndSHM-NPc1-G02 | ribophorin I [Danio rerio] | 0 | |
| ndSHM-NPc1-G08 | KIAA1560 protein [Homo sapiens] | 6.27E−38 | |
| ndSHM-NPc1-G11 | ATP synthase alpha chain, | 1.29E−23 | |
| | mitochondrial precursor | | |
| ndSHM-NPc1-H01 | similar to Tho2 [Homo sapiens] | 2.32887 | |
| | [Rattus norvegicus] | | |
| ndSHM-NPc1-H02 | Transporter, truncation | 5.24069 | up-reg E2 |
| | [Streptococcus pneumoniae R6] | | |
| ndSHM-NPc1-H03 | Hemoglobin beta chain >gi\| | 1.2944 | |
| | 7439519\|pir∥S70614 | | |
| ndSHM-NPc1-H04 | unknown | | up-reg E2 |
| ndSHM-NPc1-H05 | Cytochrome c >gi\|65467\| | 3.47E−32 | |
| | pir∥C | | |
| ndSHM-NPc1-H08 | choriogenin L (O. latipes) | 1.00E−70 | up-reg E2 |
| NONYLPHENOL | | | |
| TEST SSH | | | |
| ndSHM-NPt1-A01 | RIFIN [Plasmodium falciparum | 1.79528 | |
| | 3D7] >gi\|23498329\|e | | |
| ndSHM-NPt1-A02 | P0699H05.18 [Oryza sativa | 0.244655 | |
| | (japonica cultivar-group)] | | |
| ndSHM-NPt1-A03 | hypothetical aminotransferase | 0.421189 | |
| | [Bradyrhizobium japonicum] | | |
| ndSHM-NPt1-A04 | unknown | | up-reg E2 |
| ndSHM-NPt1-A05 | serum amyloid A protein | 9.34E−14 | up-reg E2 |
| | [Holothuria glaberrima] | | |
| ndSHM-NPt1-A08 | unknwon | | up-reg E2 |
| ndSHM-NPt1-A09 | DNAse II homolog F09G8.2 | 0.656008 | up-reg E2 |
| | [Caenorhabditis elegans] | | |
| ndSHM-NPt1-B02 | similar to peroxisomal long- | 2.30E−17 | up-reg E2 |
| | chain acyl-coA thioesterase; | | |
| | peroxisomal long-chain acyl- | | |
| | coA thioesterase ; putative | | |
| | protein [Homo sapiens] | | |
| ndSHM-NPt1-B03 | choriogenin Hminor | 1.52E−14 | up-reg E2 |
| | [Oryzias latipes] | | |
| ndSHM-NPt1-B05 | tryptophan 2,3 dioxygenase | 1.00E−60 | up-reg E2 |
| ndSHM-NPt1-B06 | ATP synthase 6 | 2.00E−23 | |
| | (Pomacentrus trilineatus) | | |
| ndSHM-NPt1-B07 | unknown | | up-reg E2 |
| ndSHM-NPt1-B11 | embyonic epidermal lectin | 4.00E−42 | up-reg E2 |
| | (X. laevis) | | |
| ndSHM-NPt1-B12 | perlecan (heparan sulfate | 2.00E−31 | |
| | proteoggllycan 2 | | |
| ndSHM-NPt1-C01 | immunoglobulin light chain | 1.38E−14 | up-reg E2 |
| | [Seriola quinqueradiata] | | |
| ndSHM-NPt1-C03 | cytochrome c oxidase subunit | 0 | up-reg E2 |
| | I [Arcos sp. KU-149] >gi\| | | |
| | 25006169\|dbj\|BAC23776.1\| | | |
| | cytochrome c oxidase subunit I | | |
| | [Arcos sp. KU-149] | | |
| ndSHM-NPt1-C05 | C9 protein | 8.96E−18 | |
| | [Oncorhynchus mykiss] | | |
| ndSHM-NPt1-C06 | pentraxin [Cyprinus carpio] | 9.55E−15 | up-reg E2 |
| ndSHM-NPt1-C09 | Very-long-chain acyl-CoA | 1.09E−13 | up-reg E2 |
| | synthetase (Very-long-chain- | | |
| | fatty-acid-CoA ligase) >gi\| | | |
| | 2645721\|gb\| | | |
| | AAB87982.1\| very-long- | | |
| | chain acyl-CoA synthetase | | |
| | [Mus musculus] | | |
| ndSHM-NPt1-C12 | dihydroorotate dehydrogenase | 5.60255 | up-reg E2 |
| | electron transfer subunit | | |
| | [Clostridium tetani | | |
| | E88] >gi\|28204415\| | | |
| | gb\|AAO36853.1\| | | |
| | dihydroorotate dehydrogenase | | |
| | electron transfer subunit | | |
| | [Clostridium tetani E88] | | |
| ndSHM-NPt1-D04 | hypothetical protein | 1.69055 | |
| | [Plasmodium yoelii yoelii] | | |
| ndSHM-NPt1-D05 | Deoxyribonuclease II precursor | 3.09E−16 | |
| | (DNase II) (Acid DNase) | | |
| | (Lysosomal DNase II) >gi\| | | |
| | 7513450\|pir∥JE0205 | | |
| | deoxyribonuclease II | | |
| | (EC 3.1.22.1) - pig >gi\| | | |
| | 3157444\|emb\|CAA04717.1\| | | |
| | Deoxyribonuclease II | | |
| | [Sus scrota] >gi\|3309153\|gb\| | | |
| | AAC39263.1\| deoxyribonuclease | | |
| | II [Sus scrofa] | | |
| ndSHM-NPt1-D07 | egg envelope protein winter | 4.00E−41 | up-reg E2 |
| | flounder | | |
| ndSHM-NPt1-D07 | similar to olfactory receptor | 4.09975 | dn-reg E2 |
| | MOR149-1 [Mus musculus] | | |
| ndSHM-NPt1-D09 | CG31752-PA [Drosophila | 1.73966 | |
| | melanogaster] >gi\| | | |
| | 22946779\|gb\| | | |
| | AAN11014.1\|AE003660_32 | | |
| | CG31752-PA | | |
| | [Drosophila melanogaster] | | |
| ndSHM-NPt1-D11 | Fibrinogen alpha (Rattus) | 5.00E−05 | up-reg field |
| ndSHM-NPt1-E02 | heparin cofactor II | 0 | |
| | [Danio rerio] | | |
| ndSHM-NPt1-E03 | FIFO-type ATP synthase | 3.32E−22 | up-reg E2 |
| | subunit g [Homo sapiens] | | |
| ndSHM-NPt1-E06 | unknown | | |
| ndSHM-NPt1-E07 | hypothetica protein XP_215519 | 5.42E−09 | |
| | [Rattus norvegicus] | | |
| ndSHM-NPt1-E12 | 6.2 kd protein [Homo | 1.75E−21 | |
| | sapiens] >gi\|12643829 | | |
| | \|sp\|Q9POU1\| | | |
| | OM07_HUMAN Probable | | |
| | mitochondrial import | | |
| | receptor subunit TOM7 homolog | | |
| | (Translocase of outer membrane | | |
| | 7 kDa subunit homolog) | | |
| | (Protein AD-014) >gi\| | | |
| | 7688665\|gb\|AAF67473.1\| | | |
| | AF150733_1 AD-014 protein | | |
| | [Homo sapiens] >gi\|12804619 | | |
| | \|gb\|AAH01732.1\|AAH01732 | | |
| | 6.2 kd protein [Homo sapiens] | | |
| ndSHM-NPt1-F01 | Hepatocyte growth factor activator | 5.12E−17 | up-reg E2 |
| | [Rattus norvegicus] | | |
| ndSHM-NPt1-F05 | Unknown | | up-reg E2 |
| ndSHM-NPt1-F07 | complement component C9 | 4.46E−34 | up-reg field |
| | [Paralichthys olivaceus] | | |
| ndSHM-NPt1-F11 | alanine-glyoxylate | 3.01E−28 | |
| | aminotransferase 2 | | |
| | [Homo sapiens] >gi\| | | |
| | 17432913\|sp\|Q9BYV1\| | | |
| | AGT2_HUMAN Alanine-glyoxylate | | |
| | aminotransferase 2, mito- | | |
| | chondrial precursor (AGT 2) | | |
| | (Beta-alanine-pyruvate | | |
| | aminotransferase) (Beta- | | |
| | ALAAT II) >gi\|12406973\| | | |
| | emb\|CAC24841.1\| alanine- | | |
| | glyoxylate aminotransferase 2 | | |
| | [Homo sapiens] | | |
| ndSHM-NPt1-G03 | KIAA1657 protein [Homo sapiens] | 8.65698 | up-reg E2 |
| ndSHM-NPt1-G07 | Unknown | | up-reg E2 |
| ndSHM-NPt1-G08 | choriogenin H [Oryzias latipes] | 3.54E−09 | up-reg E2 |
| ndSHM-NPt1-G11 | glucose-6-phosphatase, | 7.73E−09 | up-reg field |
| | catalytic; Glucose- | | |
| | 6-phosphatase [Rattus | | |
| | norvegicus] >gi\|567864 | | |
| | \|gb\|AAA74381.1\| | | |
| | glucose-6-phosphatase | | |
| ndSHM-NPt1-G12 | Orla C4 [Oryzias latipes] | 1.04E−36 | up-reg E2 |
| ndSHM-NPt1-H03 | N-acetylneuraminate pyruvate | 3.50E−17 | |
| | lyase [Mus musculus] >gi\| | | |
| | 12832930\|dbj\|BAB22314.1\| | | |
| | unnamed protein product | | |
| | [Mus musculus] >gi\| | | |
| | 18490967\|gb\|AAH22734.1\| | | |
| | RIKEN cDNA 0610033B02 gene | | |
| | [Mus musculus] >gi\| | | |
| | 26353976\|dbj\|BAC40618.1\| | | |
| | unnamed protein product | | |
| | [Mus musculus] | | |
| ndSHM-NPt1-H04 | apolipoprotein B -Atlantic salmon | 1.14E−10 | up-reg field |
| | (fragment) >gi\|854620\| | | |
| | emb\|CAA57449.1\| | | |
| | apolipoprotein B [Salmo salar] | | |
| ndSHM-NPt1-H11 | putative aryl-CoA ligase EncN | 0.513537 | |
| | [Streptomyces maritimus] | | |
| MALE/FEMALE | | | |
| UNSUBTRACTED | | | |
| SHM-D03 | cytochrome P450 | 3.00E−36 | up-reg E2; |
| | (Ictalurus punctatus) | | field |
| SHM-D02 | unknown | | up-reg E2 |
| SHM-B02 | retinol binding protein 4 | 1.00E−17 | |
| | (D. rerio) | | |
| SHM-B07 | ribosomal protein L35 (galus) | 2.00E−08 | |
| SHM-B06 | unknown | | up-reg E2 |
| SHM-B12 | Similar to 60S riboxomal | 3.00E−40 | |
| | protein L18A (D. rerio) | | |
| SHM-C03 | ribosomal protein P2 | 3.00E−21 | |
| | (I. punctatus) | | |
| SHM-C07 | C type lectins (O. mykiss) | 2.00E−11 | dn-reg E2 |
| SHM-E04 | similar to 60S ribosomal | 2.00E−15 | |
| | protein L21 | | |
| SHM-D06 | unknown protein for MGC:64127 | 6.00E−68 | up-reg E2 |
| | (D. rerio) | | |
| SHM-E01 | G protein B subunit | 2.00E−25 | |
| | (Ambystoma tigrinum) | | |
| SHM-E07 | precerebellin like protein | 7.00E−27 | |
| | (O. mykiss) | | |
| SHM-A06 | AMBP protein precursor | 3.00E−30 | |
| | microglobulin | | |
| SHM-E02 | Natural killer cel enhancement | 8.00E−31 | |
| | factor (O. mykiss) | | |
| SHM-C05 | unknown | | up-reg field |
| SHM-B10 | Similar to ribosomal protein | 1.00E−28 | |
| | L10 (D. rerio) | | |
| SHM-D12 | unknown | | |
| SHM-C01 | unknown | | |
| SHM1 | Glycosylate reductase | 3.00E−14 | |
| SHM2-1 | vitellogenin alpha (2) | in genbank | up-reg E2; EE2, |
| | | | DES, NP, MXC |
| SHM3 | vitellogenin beta(1) | in genbank | |
| SHM | Ribosomal protein S8 | 8.00E−45 | |
| SHM26 | choriogenin 3 | | |
| SHM6 | Unknown | | |
| SHM7-3 | choriogenin 2 | 1.00E−45 | |
| SHM29 | beta actin | in genbank | |
| SHM9-1 | ribosomal protein L8 | | |
| SHM74-1 | 3-hydroxy-3-methylglutaryl- | 9.00E−51 | dn-reg ES |
| | CoA reductase | | |
| SHM11 | Transferrin | | dn-reg E2; EE2, |
| | | | DES, NP, MXC |
| SHM13-1 | Low molecular mass protein 2 | 2.00E−12 | |
| SHM14 | Unknown | | |
| SHM22 | Unknown | | |
| SHM23-1 | Ribosomal protein S9 like | 6.00E−71 | |
| SHM24 | Unknown | | |
| SHM25 | Ribosomal protein S9 like | 2.00E−45 | |
| SHM39 | Unknown | | |
| SHM41 | Ubiquitin-conjugating enzyme 9 | EST match | up-reg NP |
| | | (putative) | |
| SHN42-1 | Unknown | | |
| SHM43 | Unknown protein, Acession | 4.00E−23 | |
| | numberAAH10857 | | |
| SHM48 | Unknown | | |
| SHM48-2 | Unknown | | |
| SHM51-3 | Unknown | | |
| SHM56-2 | Unknown | | |
| SHM62-2 | Hepatic lipase precursor | 7.00E−06 | |
| SHM72-3 | Coagulation Factor XI | | up-reg E2; EE2, |
| | | | DES, NP, MXC |
| SHM73 | Unknown | | |
| SHM76-2 | Alphal-microglobulin/bikunin | 1.00E−11 | d-reg E2; EE2, |
| | precursor (AMBP) protein | | DES, NP, MXC |
| | Estrogen receptor alpha | | up-reg E2; EE2, |
| | | | DES, NP, MXC, ES |

!SHEEPSHEAD? ? !LARGEMOUTH? MINNOW? ? !BASS GENES? Sequence ID? ? GENES? Sequence ID? ? !LMB#? Gene ID? Number? LMB#? Gene ID? Number

| | | | | | |
|---|---|---|---|---|---|
| LMB_COMP FACTOR | Putative | 1 | SHM IK 7A | Liver | 151 |
| Bf/C2 | complement factor | | | | |
| | Bf/C2 | | | | |
| LMB_ABMP | ABMP precursor | 2 | SHM IK 24E | Liver | 152 |
| LMB_GLUT-PEROX III | Glutathione | 3 | SHM IK 25C | Liver | 153 |
| | peroxidase III | | | | |
| LMB_Smp D1 | Small | 4 | SHM IK 5D | Liver | 154 |
| | ribonucleoprotein D1 | | | | |
| | polypeptide (16kD) | | | | |
| LMB_RIBO L6 | Ribosomal protein | 5 | SHM IKIGF-1 | Liver | 155 |
| | L6 | | | | |
| LMB_MYOSIN LIGHT | myosin regulatory | 6 | SHM IKIGF-2 | Liver | 156 |
| | light chain | | | | |
| LMB_ZPC1 | ZPC1 | 7 | ndSHM-FT1-A03 | Liver | 157 |
| LMB_CYTO-C OX 1 | Cytochrome c | 8 | ndSHM-FT1-A09 | Liver | 158 |
| | oxidase subunit I | | | | |
| LMB_LECTIN STL2 | Rhamnose binding | 9 | ndSHM-FT1-A10 | Liver | 159 |
| | lectin STL2 | | | | |
| LMB_EMAP2 | Echinoderm | 10 | ndSHM-FT1-A11 | Liver | 160 |
| | microtubule | | | | |
| | associated protein | | | | |
| | like 2 | | | | |
| LMB_ALDOLASE-B | Aldolase b | 11 | ndSHM-FT1-A12 | Liver | 161 |
| LMB_RIBO L7A | 60S ribosomal | 12 | ndSHM-FT1-B03 | Liver | 162 |
| | protein L7A | | | | |
| LMB_PROTHROMBIN | Prothrombin | 13 | ndSHM-FT1-B07 | Liver | 163 |
| | precursor | | | | |
| LMB_SSAT | SSAT | 14 | ndSHM-FT1-B10 | Liver | 164 |
| LMB_COMPLEMENT- | Complement C3 | 15 | ndSHM-FT1-C01 | Liver | 165 |
| C3 | precursor | | | | |
| LMB_RIBO L7 | Ribosomal protein | 16 | ndSHM-FT1-C03 | Liver | 166 |
| | L7 | | | | |
| LMB_H-ATPASE- | H+-ATPase subunit, | 17 | ndSHM-FT1-C04 | Liver | 167 |
| SUBUNIT | oligaomycin | | | | |
| | sensitivity conferring | | | | |
| | protein | | | | |
| LMB_RIBO L23A | Ribosomal protein | 18 | ndSHM-FT1-C09 | Liver | 168 |
| | L23a | | | | |
| LMB_ALPHA-TUBULIN | alpha tubulin | 19 | ndSHM-FT1-D06 | Liver | 169 |
| LMB_RIBO-Sa | 40S ribosomal | 20 | ndSHM-FT1-D10 | Liver | 170 |
| | protein Sa | | | | |
| LMB_VTG | Vitellogenin prcursor | 21 | ndSHM-FT1-D12 | Liver | 171 |
| LMB_NASCENT- | Nascent polypeptide- | 22 | ndSHM-FT1-E01 | Liver | 172 |
| POLYPEP | associated complex, | | | | |
| | alpha polypeptide | | | | |
| LMB_ApoH | Apoliporotein H | 23 | ndSHM-FT1-E02 | Liver | 173 |
| LMB_TBT-BP | TBT-binding protein | 24 | ndSHM-FT1-E08 | Liver | 174 |
| LMB_SOL-CAR-25A#5 | solute carrier family | 25 | ndSHM-FT1-E09 | Liver | 175 |
| | 25 alpha member 5 | | | | |
| LMB_UNNAMED- | Unnamed protein | 26 | ndSHM-FT1-E12 | Liver | 176 |
| PROTEIN | product | | | | |
| LMB_FIB-B-SUBUNIT | Fibrinogen B subunit | 27 | ndSHM-FT1-F01 | Liver | 177 |
| LMB_CIS-RETIN | cis-retinol | 28 | ndSHM-FT1-F06 | Liver | 178 |
| DEHYDRO | dehydrogenase | | | | |
| LMB_SENES-ASSOC | Putative senscence- | 29 | ndSHM-FT1-F09 | Liver | 179 |
| PROTEIN | associated protein | | | | |
| LMB_LDL RC | LDL receptor | 30 | ndSHM-FT1-F10 | Liver | 180 |
| LMB_ABC-TRANS | ABC transporter | 31 | ndSHM-FT1-F11 | Liver | 181 |
| LMB_CATHEPSIN B | Cathepsin B | 32 | ndSHM-FT1-F12 | Liver | 182 |
| LMB_SERPIN-CP9 | Serpin CP9 | 33 | ndSHM-FT1-G02 | Liver | 183 |
| LMB_TFIIIA | Transcription factor | 34 | ndSHM-FT1-G04 | Liver | 184 |
| | IIIA (TFIIIA) | | | | |
| LMB_ANTITHROMBIN | Antithrombin III | 35 | ndSHM-FT1-G08 | Liver | 185 |
| III | | | | | |
| LMB_RIKEN | RIKEN cDNA | 36 | ndSHM-FT1-H02 | Liver | 186 |
| 1810056020 | 1810056020 | | | | |
| LMB_WEE-I | Wee I tyrosine | 37 | ndSHM-FT1-H03 | Liver | 187 |
| | kinase | | | | |
| LMB_HAPTOGLOBIN | Haptoglobin | 38 | ndSHM-FT1-H04 | Liver | 188 |
| LMB_APOA-I | APOPLIPOPROTEIN | 39 | ndSHM-FT1-H06 | Liver | 189 |
| | A-I | | | | |
| LMB_ALPHA-1 | alpha -1 antitrypsin | 40 | ndSHM-FT1-H07 | Liver | 190 |
| ANTITRYPSIN | homolog precursor | | | | |
| LMB_APOE | Apolipoprotein E | 41 | ndSHM-FT1-H10 | Liver | 191 |
| LMB_ZPC4 | ZPC4 | 42 | ndSHM-FT1-H11 | Liver | 192 |
| LMB_LECTIN 9 | C-type lectin | 43 | ndSHM-FT1-H12 | Liver | 193 |
| | superfamily 9 | | | | |
| LMB_ATPASE 6 | ATPase subunit 6 | 44 | ndSHM-MC1-A02 | Liver | 194 |
| LMB_ITI | inter-alpha-trypsin | 45 | ndSHM-MC1-A03 | Liver | 195 |
| | inhibitor “ITI” | | | | |
| LMB_EIF-3#7 | Eukaryotic | 46 | ndSHM-MC1-A04 | Liver | 196 |
| | translation initiation | | | | |
| | factor 3 subunit 7 | | | | |
| LMB_HEPCIDIN | Hepcidin precursor | 47 | ndSHM-MC1-A05 | Liver | 197 |
| LMB_PTTP | Pituitary tumor | 48 | ndSHM-MC1-A07 | Liver | 198 |
| | transforming protein | | | | |
| LMB_TOXIN-1 | Toxin-1 | 49 | ndSHM-MC1-A09 | Liver | 199 |
| LMB_COAG FACTOR | Coagulation factor | 50 | ndSHM-MC1-A11 | Liver | 200 |
| VII | VII | | | | |
| LMB_CDC42-2 | cdc 42 isoform 2 | 51 | ndSHM-MC1-B01 | Liver | 201 |
| LMB_WARM-WATER | Warm water | 52 | ndSHM-MC1-B03 | Liver | 202 |
| ACC PROTEIN | acclimation-related | | | | |
| | protein | | | | |
| LMB_CYTO-C OX II | Cytochrome c | 53 | ndSHM-MC1-B04 | Liver | 203 |
| | oxidase subunit II | | | | |
| LMB_L10A | 60S ribosomal | 54 | ndSHM-MC1-B05 | Liver | 204 |
| | protein L10A | | | | |
| LMB_KALLIKREIN | Kallikrein | 55 | ndSHM-MC1-B08 | Liver | 205 |
| LMB_DANIO EST | Danio EST | 56 | ndSHM-MC1-B10 | Liver | 206 |
| 3818635 | IMAGE: 3818635 | | | | |
| LMB_ALPHA-2- | alpha-2- | 57 | ndSHM-MC1-B11 | Liver | 207 |
| MACROGLOB-1 | macroglobulin-1 | | | | |
| LMB_HAPTOGLOB | Haptoglobin-related | 58 | ndSHM-MC1-C02 | Liver | 208 |
| RELATED PROT | protein | | | | |
| LMB_FILAMEN-B | Filamen B | 59 | ndSHM-MC1-C04 | Liver | 209 |
| LMB_UBIQUITIN | ubiquitin | 60 | ndSHM-MC1-C05 | Liver | 210 |
| LMB_RXR INTERACT | Retinoid X receptor | 61 | ndSHM-MC1-C08 | Liver | 211 |
| PROT | interacting protein | | | | |
| LMB_MITOCHON-ATP- | ATP synthase alpha | 62 | ndSHM-MC1-C10 | Liver | 212 |
| SYNTHASE | chain mitochondrial | | | | |
| | precursor | | | | |
| LMB_TATA BOX BP | TATA-box binding | 63 | ndSHM-MC1-D01 | Liver | 213 |
| | protein | | | | |
| LMB_DIFF-REG | Diiferentially | 64 | ndSHM-MC1-D02 | Liver | 214 |
| TROUT PROT-1 | regulated trout | | | | |
| | protein 1 | | | | |
| LMB_LIVER-REGEN- | liver regeneration | 65 | ndSHM-MC1-D03 | Liver | 215 |
| REL PROT | related protein | | | | |
| LMB_SERPIN-2B | Serpin 2b | 66 | ndSHM-MC1-D04 | Liver | 216 |
| LMB_APO-A1 | Apolipoprotein A-I-1 | 67 | ndSHM-MC1-D06 | Liver | 217 |
| | precursor | | | | |
| LMB_M-PHASE PROT | M-phase | 68 | ndSHM-MC1-D10 | Liver | 218 |
| 6 | phosphoprotein 6 | | | | |
| LMB_PROSTAGLAND- | Prostaglandin D | 69 | ndSHM-MC1-E01 | Liver | 219 |
| D-SYNTHASE | synthase-like protein | | | | |
| | (lipocalin type) | | | | |
| LMB_LYRIC | LYRIC | 70 | ndSHM-MC1-E05 | Liver | 220 |
| LMB_CYSTATIN-PREC | Cystatin precursor | 71 | ndSHM-MC1-E06 | Liver | 221 |
| LMB_RIKEN 2700038 | RIKEN cDNA | 72 | ndSHM-MC1-E08 | Liver | 223 |
| | 2700038 | | | | |
| LMB_DIAZEPAM- | Membrane | 73 | ndSHM-MC1-E10 | Liver | 224 |
| BINDING INHIB | associated | | | | |
| | diazepam-binding | | | | |
| | inhibitor | | | | |
| LMB_IGF-I | IGF-I | 74 | ndSHM-MC1-E12 | Liver | 225 |
| LMB_ESTP4_D11 | ESTP4_D11 | 75 | ndSHM-MC1-F01 | Liver | 226 |
| LMB_ESTDHT_6 | ESTDHT_6 | 76 | ndSHM-MC1-F02 | Liver | 227 |
| LMB_ESTDHT_7 | ESTDHT_7 | 77 | ndSHM-MC1-F03 | Liver | 228 |
| LMB_ESTDHT_13 | ESTDHT_13 | 78 | ndSHM-MC1-F06 | Liver | 229 |
| LMB_ESTDHT_50 | ESTDHT_50 | 79 | ndSHM-MC1-F11 | Liver | 230 |
| LMB_ESTDHT_51 | ESTDHT_51 | 80 | ndSHM-MC1-F12 | Liver | 231 |
| LMB_ESTDHT_53 | ESTDHT_53 | 81 | ndSHM-MC1-G01 | Liver | 232 |
| LMB_ESTDHT_60 | ESTDHT_60 | 82 | ndSHM-MC1-G02 | Liver | 233 |
| LMB_ESTDHT_62 | ESTDHT_62 | 83 | ndSHM-MC1-G03 | Liver | 234 |
| LMB_ESTDHT_68 | ESTDHT_68 | 84 | ndSHM-MC1-G04 | Liver | 235 |
| LMB_ESTDHT_69 | ESTDHT_69 | 85 | ndSHM-MC1-G05 | Liver | 236 |
| LMB_ESTP4_A02 | ESTP4_A02 | 86 | ndSHM-MC1-G07 | Liver | 237 |
| LMB_ESTP4_B03 | ESTP4_B03 | 87 | ndSHM-MC1-G11 | Liver | 238 |
| LMB_ESTP4_B04 | ESTP4_B04 | 88 | ndSHM-MC1-G12 | Liver | 239 |
| LMB_ESTP4_B07 | ESTP4_B07 | 89 | ndSHM-MC1-H02 | Liver | 240 |
| LMB_ESTP4_B08 | ESTP4_B08 | 90 | ndSHM-MC1-H03 | Liver | 241 |
| LMB_ESTP4_B09 | ESTP4_B09 | 91 | ndSHM-MC1-H04 | Liver | 242 |
| LMB_ESTP4_C03 | ESTP4_C03 | 92 | ndSHM-MC1-H06 | Liver | 243 |
| LMB_ESTP4_C04 | ESTP4_C04 | 93 | ndSHM-MC1-H08 | Liver | 244 |
| LMB_ESTP4_C06 | ESTP4_C06 | 94 | ndSHM-MC1-H09 | Liver | 245 |
| LMB_ESTP4_D04 | ESTP4_D04 | 95 | ndSHM-MC1-H10 | Liver | 246 |
| LMB_ESTP4_D08 | ESTP4_D08 | 96 | ndSHM-MC1-H12 | Liver | 247 |
| LMB_ESTP4_D10 | ESTP4_D10 | 97 | ndSHM-MT1-A02 | Liver | 248 |
| LMB_ESTP4_E01 | ESTP4_E01 | 98 | ndSHM-MT1-A03 | Liver | 248 |
| LMB_ESTP4_E03 | ESTP4_E03 | 99 | ndSHM-MT1-A05 | Liver | 249 |
| LMB_ESTP4_E06 | ESTP4_E06 | 100 | ndSHM-MT1-B09 | Liver | 250 |
| LMB_ESTP4_E08 | ESTP4_E08 | 101 | ndSHM-MT1-C05 | Liver | 251 |
| LMB_ESTP4_E12 | ESTP4_E12 | 102 | ndSHM-MT1-C08 | Liver | 252 |
| LMB_ESTP4_F06 | ESTP4_F06 | 103 | ndSHM-MT1-D04 | Liver | 253 |
| LMB_ESTP4_G06 | ESTP4_G06 | 104 | ndSHM-MT1-D05 | Liver | 254 |
| LMB_ESTP4_G11 | ESTP4_G11 | 105 | ndSHM-MT1-D07 | Liver | 255 |
| LMB_ESTP4_H02 | ESTP4_H02 | 106 | ndSHM-MT1-E02 | Liver | 256 |
| LMB_ESTP4_H04 | ESTP4_H04 | 107 | ndSHM-MT1-E03 | Liver | 257 |
| LMB_ESTP4_H05 | ESTP4_H05 | 108 | ndSHM-MT1-E05 | Liver | 258 |
| LMB_ESTP4_H07 | ESTP4_H07 | 109 | ndSHM-MT1-F11 | Liver | 259 |
| LMB_ESTP4_H08 | ESTP4_H08 | 110 | ndSHM-MT1-G03 | Liver | 260 |
| LMB_EST- | EST- | 111 | ndSHM-MT1-H05 | Liver | 261 |
| SEASONAL_02 | SEASONAL_02 | | | | |
| LMB_EST- | EST- | 112 | ndSHM-MXCc1-A04 | Liver | 262 |
| SEASONAL_03 | SEASONAL_03 | | | | |
| LMB_EST- | EST- | 113 | ndSHM-MXCc1-A09 | Liver | 263 |
| SEASONAL_04 | SEASONAL_04 | | | | |
| LMB_EST- | EST- | 114 | ndSHM-MXCc1-A10 | Liver | 264 |
| SEASONAL_06 | SEASONAL_06 | | | | |
| LMB_EST- | EST- | 115 | ndSHM-MXCc1-A11 | Liver | 265 |
| SEASONAL_09 | SEASONAL_09 | | | | |
| LMB_EST- | EST- | 116 | ndSHM-MXCc1-B02 | Liver | 266 |
| SEASONAL_11 | SEASONAL_11 | | | | |
| LMB_EST- | EST- | 117 | ndSHM-MXCc1-B03 | Liver | 267 |
| SEASONAL_12 | SEASONAL_12 | | | | |
| LMB_EST-SEASONAL- | EST-SEASONAL-14 | 118 | ndSHM-MXCc1-B04 | Liver | 268 |
| 14 | | | | | |
| LMB_EST- | EST- | 119 | ndSHM-MXCc1-B06 | Liver | 269 |
| SEASONAL_16 | SEASONAL_16 | | | | |
| LMB_EST- | EST- | 120 | ndSHM-MXCc1-B07 | Liver | 270 |
| SEASONAL_17 | SEASONAL_17 | | | | |
| LMB_EST- | EST- | 121 | ndSHM-MXCc1-B08 | Liver | 271 |
| SEASONAL_22 | SEASONAL_22 | | | | |
| LMB_EST- | EST- | 122 | ndSHM-MXCc1-C04 | Liver | 272 |
| SEASONAL_51 | SEASONAL_51 | | | | |
| LMB_EST- | EST- | 123 | ndSHM-MXCc1-C06 | Liver | 273 |
| SEASONAL_52 | SEASONAL_52 | | | | |
| LMB_EST- | EST- | 124 | ndSHM-MXCc1-C11 | Liver | 274 |
| SEASONAL_54 | SEASONAL_54 | | | | |
| LMB_EST- | EST- | 125 | ndSHM-MXCc1-D03 | Liver | 275 |
| SEASONAL_55 | SEASONAL_55 | | | | |
| LMB_EST- | EST- | 126 | ndSHM-MXCc1-D04 | Liver | 276 |
| SEASONAL_56 | SEASONAL_56 | | | | |
| LMB_EST-- | EST- | 127 | ndSHM-MXCc1-D05 | Liver | 277 |
| SEASONAL_58 | SEASONAL_58 | | | | |
| LMB_EST-- | EST- | 128 | ndSHM-MXCc1-D08 | Liver | 278 |
| SEASONAL_59 | SEASONAL_59 | | | | |
| LMB_EST-- | EST- | 129 | ndSHM-MXCc1-D10 | Liver | 279 |
| SEASONAL_61 | SEASONAL_61 | | | | |
| LMB_EST-- | EST- | 130 | ndSHM-MXCc1-D12 | Liver | 280 |
| SEASONAL_62 | SEASONAL_62 | | | | |
| LMB_EST-- | EST- | 131 | ndSHM-MXCc1-E01 | Liver | 281 |
| SEASONAL_64 | SEASONAL_64 | | | | |
| LMB_EST-- | EST- | 132 | ndSHM-MXCc1-E09 | Liver | 282 |
| SEASONAL_68 | SEASONAL_68 | | | | |
| LMB_EST-- | EST- | 133 | ndSHM-MXCc1-E11 | Liver | 283 |
| SEASONAL_70 | SEASONAL_70 | | | | |
| LMB_EST-- | EST- | 134 | ndSHM-MXCc1-F01 | Liver | 284 |
| SEASONAL_71 | SEASONAL_71 | | | | |
| LMB_EST-- | EST- | 135 | ndSHM-MXCc1-F03 | Liver | 285 |
| SEASONAL_72 | SEASONAL_72 | | | | |
| LMB_EST-- | EST- | 136 | ndSHM-MXCc1-F07 | Liver | 286 |
| SEASONAL_75 | SEASONAL_75 | | | | |
| LMB_EST-- | EST- | 137 | ndSHM-MXCc1-F10 | Liver | 287 |
| SEASONAL_77 | SEASONAL_77 | | | | |
| LMB_EST-- | EST- | 138 | ndSHM-MXCc1-G02 | Liver | 288 |
| SEASONAL_85 | SEASONAL_85 | | | | |
| LMB_EST-- | EST- | 139 | ndSHM-MXCc1-G03 | Liver | 289 |
| SEASONAL_88 | SEASONAL_88 | | | | |
| LMB_EST-- | EST- | 140 | ndSHM-MXCc1-G04 | Liver | 290 |
| SEASONAL_90 | SEASONAL_90 | | | | |
| LMB_EST-- | EST- | 141 | ndSHM-MXCc1-G12 | Liver | 291 |
| SEASONAL_92 | SEASONAL_92 | | | | |
| LMB_EST-- | EST- | 142 | ndSHM-MXCc1-H05 | Liver | 292 |
| SEASONAL_97 | SEASONAL_97 | | | | |
| LMB_EST-- | EST- | 143 | ndSHM-MXCc1-H09 | Liver | 293 |
| SEASONAL_F11 | SEASONAL_F11 | | | | |
| LMB_EST-- | EST- | 144 | ndSHM-MXCt1-B05 | Liver | 294 |
| SEASONAL_F17 | SEASONAL_F17 | | | | |
| LMB_EST-- | EST- | 145 | ndSHM-MXCt1-B08 | Liver | 295 |
| SEASONAL_F21 | SEASONAL_F21 | | | | |
| LMB_ER-ALPHA | ESTROGEN | 146 | ndSHM-MXCt1-C02 | Liver | 296 |
| | RECEPTOR ALPHA | | | | |
| LMB_ER-BETA | ESTROGEN | 147 | ndSHM-MXCt1-C11 | Liver | 297 |
| | RECEPTOR BETA | | | | |
| LMB_ER-GAMMA | ESTROGEN | 148 | ndSHM-MXCt1-D09 | Liver | 298 |
| | RECEPTOR | | | | |
| | GAMMA | | | | |
| LMB_STAR | STAR PROTEIN | 149 | ndSHM-MXCt1-E04 | Liver | 299 |
| LMB_SF1 | SF1 PROTEIN | 150 | ndSHM-MXCt1-E06 | Liver | 300 |
| | FRAGMENT | | | | |
| LMB1-3 | | 420 | ndSHM-MXCt1-E09 | Liver | 301 |
| LMB2-2 | | 421 | ndSHM-MXCt1-F11 | Liver | 302 |
| LMB3-1 | | 422 | ndSHM-MXCt1-E12 | Liver | 303 |
| LMB4-1 | | 423 | ndSHM-MXCt1-G03 | Liver | 304 |
| LMB5 | | 424 | ndSHM-MXCt1-H03 | Liver | 305 |
| LMB6-1 | | 425 | ndSHM-NPc1-A12 | Liver | 306 |
| LMB7-1 | | 426 | ndSHM-NPc1-B01 | Liver | 307 |
| LMB8-2 | | 427 | ndSHM-NPc1-B08 | Liver | 308 |
| LMB9-1 | | 428 | ndSHM-NPc1-B09 | Liver | 309 |
| LMB10-1 | | 429 | ndSHM-NPc1-C04 | Liver | 310 |
| LMB11-2 | | 430 | ndSHM-NPc1-C06 | Liver | 311 |
| LMB12-1 | | 431 | ndSHM-NPc1-C11 | Liver | 312 |
| LMB13-2 | | 432 | ndSHM-NPc1-E01 | Liver | 313 |
| LMB14-1 | | 433 | ndSHM-NPc1-E06 | Liver | 314 |
| LMB15-1 | | 434 | ndSHM-NPc1-E11 | Liver | 315 |
| LMB16-2 | | 435 | ndSHM-NPc1-F01 | Liver | 316 |
| LMB17-2 | | 436 | ndSHM-NPc1-F05 | Liver | 316 |
| LMB18-3 | | 437 | ndSHM-NPc1-F06 | Liver | 318 |
| LMB19-1 | | 438 | ndSHM-NPc1-F07 | Liver | 319 |
| LMB20-1 | | 439 | ndSHM-NPc1-F08 | Liver | 320 |
| LMB21-1 | | 440 | ndSHM-NPc1-G02 | Liver | 321 |
| LMB22-1 | | 441 | ndSHM-NPc1-G08 | Liver | 322 |
| LMB23-1 | | 442 | ndSHM-NPc1-G11 | Liver | 323 |
| LMB24-1 | | 443 | ndSHM-NPc1-H01 | Liver | 324 |
| LMB25-1 | | 444 | ndSHM-NPc1-H02 | Liver | 325 |
| LMB26-1 | | 445 | ndSHM-NPc1-H03 | Liver | 326 |
| LMB27-1 | | 446 | ndSHM-NPc1-H04 | Liver | 327 |
| LMB28-2 | | 447 | ndSHM-NPc1-H05 | Liver | 328 |
| LMB29-2 | | 448 | ndSHM-NPc1-H08 | Liver | 329 |
| LMB30-1/Forward | | 449 | ndSHM-NPt1-A01 | Liver | 330 |
| LMB30-1/Reverse | | 450 | | | |
| LMB31 | | 451 | ndSHM-NPt1-A02 | Liver | 331 |
| LMB32-1 | | 452 | ndSHM-NPt1-A03 | Liver | 332 |
| LMB33-1/A | | 453 | ndSHM-NPt1-A04 | Liver | 333 |
| LMB33-1/B | | 454 | | | |
| LMB34-1 | | 455 | ndSHM-NPt1-A05 | Liver | 334 |
| LMB35-1 | | 456 | ndSHM-NPt1-A08 | Liver | 335 |
| LMB36-1 | | 457 | ndSHM-NPt1-A09 | Liver | 336 |
| LMB37-1/A | | 458 | ndSHM-NPt1-B02 | Liver | 337 |
| LMB37-1/B | | 459 | | | |
| LMB38-1 | | 460 | ndSHM-NPt1-B03 | Liver | 338 |
| LMB39-1 | | 461 | ndSHM-NPt1-B05 | Liver | 339 |
| LMB40-1 | | 462 | ndSHM-NPt1-B06 | Liver | 340 |
| LMB41-1 | | 463 | ndSHM-NPt1-B07 | Liver | 341 |
| LMB42-1 | | 464 | ndSHM-NPt1-B11 | Liver | 342 |
| LMB43-1 | | 465 | ndSHM-NPt1-B12 | Liver | 343 |
| LMB44-1 | | 466 | ndSHM-NPt1-C01 | Liver | 344 |
| LMB45-1/Forward | | 467 | ndSHM-NPt1-C03 | Liver | 345 |
| LMB45-1/Reverse | | 468 | | | |
| LMB46-1 | | 469 | ndSHM-NPt1-C05 | Liver | 346 |
| LMB47-2 | | 470 | ndSHM-NPt1-C06 | Liver | 347 |
| LMB48-1 | | 471 | ndSHM-NPt1-C09 | Liver | 348 |
| LMB49-1 | | 472 | ndSHM-NPt1-C12 | Liver | 349 |
| LMB50-1 | | 473 | ndSHM-NPt1-D04 | Liver | 350 |
| LMB51-1 | | 474 | ndSHM-NPt1-D05 | Liver | 351 |
| LMB52-1 | | 475 | ndSHM-NPt1-D07 | Liver | 352 |
| LMB53-1 | | 476 | ndSHM-NPt1-D07 | Liver | 353 |
| LMB54-2 | | 477 | ndSHM-NPt1-D09 | Liver | 354 |
| LMB55-1 | | 478 | ndSHM-NPt1-D11 | Liver | 355 |
| LMB56-1 | | 479 | ndSHM-NPt1-E02 | Liver | 356 |
| LMB57 | | 480 | ndSHM-NPt1-E03 | Liver | 357 |
| LMB58-1 | | 481 | ndSHM-NPt1-E06 | Liver | 358 |
| LMB59-1 | | 482 | ndSHM-NPt1-E07 | Liver | 359 |
| LMB60-1 | | 483 | ndSHM-NPt1-E12 | Liver | 360 |
| LMB61-1 | | 484 | ndSHM-NPt1-F01 | Liver | 361 |
| LMB63-1 | | 485 | ndSHM-NPt1-F05 | Liver | 362 |
| LMB64-1 | | 486 | ndSHM-NPt1-F07 | Liver | 363 |
| LMB65-1 | | 487 | ndSHM-NPt1-F11 | Liver | 364 |
| LMB66-1 | | 488 | ndSHM-NPt1-G03 | Liver | 365 |
| LMB67-1 | | 489 | ndSHM-NPt1-G07 | Liver | 366 |
| LMB68-1 | | 490 | ndSHM-NPt1-G08 | Liver | 367 |
| LMB69-2 | | 491 | ndSHM-NPt1-G11 | Liver | 368 |
| LMB70-2 | | 492 | ndSHM-NPt1-G12 | Liver | 369 |
| LMB71-1 | | 493 | ndSHM-NPt1-H03 | Liver | 370 |
| LMB72-1 | | 494 | ndSHM-NPt1-H04 | Liver | 371 |
| LMB73-1 | | 495 | ndSHM-NPt1-H11 | Liver | 372 |
| LMB74-3 | | 496 | SHM-D03 | Liver | 373 |
| LMB75-1 | | 497 | SHM-D02 | Liver | 374 |
| LMB76-2 | | 498 | SHM-B02 | Liver | 375 |
| LMB77-1 | | 499 | SHM-B07 | Liver | 376 |
| LMB78-1 | | 500 | SHM-B06 | Liver | 377 |
| LMB79-2 | | 501 | SHM-B12 | Liver | 378 |
| LMB80-1 | | 502 | SHM-C03 | Liver | 379 |
| LMB81-1 | | 503 | SHM-C07 | Liver | 380 |
| LMB82-1 | | 504 | SHM-E04 | Liver | 381 |
| LMB83 | | 505 | SHM-D06 | Liver | 382 |
| LMB84-1 | | 506 | SHM-E01 | Liver | 383 |
| LMB85-1 | | 507 | SHM-E07 | Liver | 384 |
| LMB86-1 | | 508 | SHM-A06 | Liver | 385 |
| LMB87 | | 509 | SHM-E02 | Liver | 386 |
| LMB88-1 | | 510 | SHM-C05 | Liver | 387 |
| LMB89-1 | | 511 | SHM-B10 | Liver | 388 |
| LMB90-1 | | 512 | SHM-D12 | Liver | 389 |
| LMB91-1 | | 513 | SHM-C01 | Liver | 390 |
| LMB92-1 | | 514 | SHM1 | | 391 |
| LMB93-1 | | 515 | SHM2-1 | | 392 |
| LMB94-1 | | 516 | SHM3 | | 393 |
| LMB95-1 | | 517 | | | |
| LMB96-1 | | 518 | SHM26 | | 394 |
| LMB97 | | 519 | SHM6 | | 395 |
| LMB98-1 | | 520 | SHM7-3 | | 396 |
| LMB99-1 | | 521 | SHM29 | | 397 |
| LMB100-1 | | 522 | SHM9-1 | | 398 |
| LMB101-1 | | 523 | SHM74-1 | | 399 |
| LMB102-1 | | 524 | SHM11 | | 400 |
| LMB103-1 | | 525 | SHM13-1 | | 401 |
| LMB104-3 | | 526 | SHM14 | | 402 |
| LMB105-1 | | 527 | SHM-18 | | 403 |
| LMB107-1 | | 528 | SHM22 | | 404 |
| LMB108-1 | | 529 | SHM23-1 | | 405 |
| LMB109-1 | | 530 | SHM24 | | 406 |
| LMB110-1 | | 531 | SHM25 | | 407 |
| LMB112-1 | | 532 | SHM39 | | 408 |
| LMB116-1 | | 533 | SHM41 | | 409 |
| LMB118-1 | | 534 | SHN42-1 | | 410 |
| LMB120-1 | | 535 | SHM43 | | 411 |
| LMB121-1 | | 536 | SHM48 | | 412 |
| LMB122-1 | | 537 | SHM48-2 | | 413 |
| LMB123-1 | | 538 | SHM51-3 | | 414 |
| LMB128-1 | | 539 | SHM56-2 | | 415 |
| LMB129-1 | | 540 | SHM62-2 | | 416 |
| LMB130-1 | | 541 | SHM72-3 | | 417 |
| LMB132-1 | | 542 | SHM73 | | 418 |
| LMB133-1 | | 543 | SHM76-2 | | 419 |
| LMB134-3 | | 544 | | | |
| LMB135 | | 545 | | | |
| LMB136-1 | | 546 | | | |
| LMB137-2 | | 546 | | | |
| LMB138-1 | | 547 | | | |
| LMB139-1 | | 549 | | | |
| LMB140-1 | | 550 | | | |
| LMB141 | | 551 | | | |
| LMB142-1 | | 552 | | | |
| LMB144-1 | | 553 | | | |
| LMB150 | | 554 | | | |
| LMB151 | | 555 | | | |
| LMB_ESTP4-E01 | | 556 | | | |
| LMB_ESTDHT64 | | 557 | | | |
| LMB_LIV-REGER-PROT | | 558 | | | |
| LMB_RIKEN 1110001M01 | | 559 | | | |
| LMB_EST-SEASONALf17 | | 560 | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting the presence of an agent having estrogenic or androgenic activity in a sample, the method comprising the steps of:
(A) providing at least one fish cell which was exposed to the sample;
(B) analyzing the at least one fish cell for expression of at least one gene wholly or partially encoded by a nucleotide sequence selected from the group consisting of SEQ ID NOs: 1-560; and
(C) comparing the expression of the at least one gene in the cell compared to the expression of the at least gene in a control cell not exposed to the sample or an agent having estrogenic or androgenic activity, wherein a difference in the expression of the at least one gene in the at least one fish cell compared to the expression of the at least one gene in the control cell indicates that the sample contains an agent having estrogenic or androgenic activity.

2. The method of claim 1, wherein the step of analyzing the at least one fish cell for expression of at least one gene comprises analyzing the cell for expression of at least two different genes each being wholly or partially encoded by a nucleotide sequence selected from the group consisting of SEQ ID NOs: 1-560.

3. The method of claim 1, wherein the step of analyzing the at least one fish cell for expression of at least one gene comprises analyzing the cell for expression of at least three different genes each being wholly or partially encoded by a nucleotide sequence selected from the group consisting of SEQ ID NOs: 1-560.

4. The method of claim 1, wherein the step of analyzing the at least one fish cell for expression of at least one gene comprises analyzing the cell for expression of at least four different genes each being wholly or partially encoded by a nucleotide sequence selected from the group consisting of SEQ ID NOs: 1-560.

5. The method of claim 1, wherein the step of analyzing the at least one fish cell for expression of at least one gene comprises analyzing the cell for expression of at least ten different genes each being wholly or partially encoded by a nucleotide sequence selected from the group consisting of SEQ ID NOs: 1-560.

6. The method of claim 1, wherein the step of analyzing the at least one fish cell for expression of at least one gene comprises analyzing the cell for expression of at least twenty-five different genes each being wholly or partially encoded by a nucleotide sequence selected from the group consisting of SEQ ID NOs: 1-560.

7. The method of claim 1, wherein the step of analyzing the at least one fish cell for expression of at least one gene comprises analyzing the cell for expression of at least one hundred different genes each being wholly or partially encoded by a nucleotide sequence selected from the group consisting of SEQ ID NOs: 1-560.

8. The method of claim 1, wherein the at least one fish cell is a large mouth bass cell.

9. The method of claim 1, wherein the at least one fish cell is a sheep's head minnow cell.

10. The method of claim 1, wherein the at least one fish cell was obtained from a fish that had been exposed to the sample.

11. The method of claim 1, wherein the step of analyzing the at least one fish cell for expression of at least one gene comprises isolating RNA transcripts from the at least one cell.

12. The method of claim 11, wherein the step of analyzing the at least one fish cell for expression of at least one gene further comprises contacting the isolated RNA transcripts or nucleic acids derived therefrom using the isolated RNA transcripts as templates with at least one probe that hybridizes under stringent hybridization conditions to at least one nucleotide sequence selected from the group consisting of SEQ ID NOs: 1-560.

13. The method of claim 12, wherein the at least one probe is immobilized on a substrate.

14. The method of claim 13, wherein the substrate is comprised of a substance selected from the group consisting of: nylon, nitrocellulose, glass, and plastic.

15. The method of claim 11, wherein the step of analyzing the at least one fish cell for expression of at least one gene further comprises contacting the isolated RNA transcripts or nucleic acids derived therefrom using the isolated RNA transcripts as templates with at least two different probes that each hybridize under stringent hybridization conditions to a different nucleotide sequence selected from the group consisting of SEQ ID NOs: 1-560.

16. The method of claim 11, wherein the step of analyzing the at least one fish cell for expression of at least one gene further comprises contacting the isolated RNA transcripts or nucleic acids derived therefrom using the isolated RNA transcripts as templates with at least three different probes that each hybridize under stringent hybridization conditions to a different nucleotide sequence selected from the group consisting of SEQ ID NOs: 1-560.

17. The method of claim 11, wherein the step of analyzing the at least one fish cell for expression of at least one gene further comprises contacting the isolated RNA transcripts or nucleic acids derived therefrom using the isolated RNA transcripts as templates with at least four different probes that each hybridize under stringent hybridization conditions to a different nucleotide sequence selected from the group consisting of SEQ ID NOs: 1-560.

18. The method of claim 11, wherein the step of analyzing the at least one fish cell for expression of at least one gene further comprises contacting the isolated RNA transcripts or nucleic acids derived therefrom using the isolated RNA transcripts as templates with at least ten different probes that each hybridize under stringent hybridization conditions to a different nucleotide sequence selected from the group consisting of SEQ ID NOs: 1-560.

19. The method of claim 11, wherein the step of analyzing the at least one fish cell for expression of at least one gene further comprises contacting the isolated RNA transcripts or nucleic acids derived therefrom using the isolated RNA transcripts as templates with at least twenty-five different probes that each hybridize under stringent hybridization conditions to a different nucleotide sequence selected from the group consisting of SEQ ID NOs: 1-560.

20. The method of claim 11, wherein the step of analyzing the at least one fish cell for expression of at least one gene further comprises contacting the isolated RNA transcripts or nucleic acids derived therefrom using the isolated RNA transcripts as templates with at least one-hundred different probes that each hybridize under stringent hybridization conditions to a different nucleotide sequence selected from the group consisting of SEQ ID NOs: 1-560.

21. The method of claim 12, wherein the at least one probe is conjugated with a detectable label.

22. The method of claim 21, wherein the isolated RNA transcripts or nucleic acids derived therefrom are conjugated with a detectable label.

23. The method of claim 1, further comprising analyzing the control cell not exposed to the sample or an agent having estrogenic or androgenic activity for expression of at least one gene wholly or partially encoded by a nucleotide sequence selected from the group consisting of SEQ ID NOs: 1-560.

24. The method of claim 23, wherein the step of analyzing the control cell for expression of at least one gene further comprises isolating RNA transcripts from the control cell and contacting the isolated RNA transcripts or nucleic acids derived therefrom using the isolated RNA transcripts as templates with at least one probe that hybridizes under stringent hybridization conditions to at least one nucleotide sequence selected from the group consisting of SEQ ID NOs: 1-560.

25. The method of claim 24, wherein the RNA transcripts or nucleic acids derived therefrom isolated from the at least one fish cell are conjugated with a first detectable label and the RNA transcripts or nucleic acids derived therefrom isolated from the control cell are conjugated with a second detectable label differing from the first detectable label.

26. The method of claim 23, further comprising isolating RNA transcripts from the at least one fish cell and contacting the RNA transcripts isolated from the at least one fish cell or nucleic acids derived therefrom using the RNA transcripts isolated from the at least one fish cell as templates with at least one molecule that hybridizes under stringent conditions to at least one nucleotide sequence selected from the group consisting of SEQ ID NOs: 1-560.

27. The method of claim 26, wherein the at least one probe is conjugated with a first detectable label and the at least one molecule is conjugated with a second detectable label differing in chemical structure from the first detectable label.

28. The method of claim 27, wherein the step of comparing the expression of the at least one nucleic acid in the cell compared to the expression of the at least one nucleic acid in a control cell not exposed to the sample or an agent having estrogenic or androgenic activity comprises quantifying the amount of first detectable label associated with the RNA transcripts isolated from the control cell or nucleic acids derived therefrom, and quantifying the amount of second detectable label associated with the RNA transcripts isolated from the at least one fish cell or nucleic acids derived therefrom.

29. The method of claim 1, further comprising the step of contacting the at least one fish cell with the sample prior to the step of analyzing the at least one fish cell for expression of the at least one gene.

30. The method of claim 1, wherein the sample comprises water.

31. The method of claim 1, further comprising the steps of:
providing a fish;
contacting the fish with the sample; and
isolating the at least one fish cell from the fish contacted with the sample.

32. A method for determining whether an agent has estrogenic, anti-estrogenic, androgenic or anti-androgenic activity, the method comprising the steps of:
providing at least one fish cell;
contacting the at least one fish cell with the agent;
analyzing the at least one fish cell for expression of at least one gene wholly or partially encoded by a nucleotide sequence selected from the group consisting of SEQ ID NOs: 1-560; and
comparing the expression of the at least one gene in the cell compared to the expression of the at least one nucleic acid in a control cell not exposed to the sample or an agent having estrogenic or androgenic activity, wherein a difference in the expression of the at least one nucleic acid in the at least one fish cell compared to the expression of the at least one nucleic acid in the control cell indicates that the agent has estrogenic, anti-estrogenic, androgenic, or anti-androgenic activity.

33. A substrate having immobilized thereon at least one nucleic acid comprising a nucleotide sequence selected from the group consisting of SEQ ID NOs: 1-560 and complements thereof.

34. The substrate of claim 33, wherein the substrate has immobilized thereon at least two different nucleic acids each comprising a different nucleotide sequence selected from the group consisting of SEQ ID NOs: 1-560 and complements thereof.

35. The substrate of claim 33, wherein the substrate has immobilized thereon at least three different nucleic acids each comprising a different nucleotide sequence selected from the group consisting of SEQ ID NOs: 1-560 and complements thereof.

36. The substrate of claim 33, wherein the substrate has immobilized thereon at least four different nucleic acids each comprising a different nucleotide sequence selected from the group consisting of SEQ ID NOs: 1-560 and complements thereof.

37. The substrate of claim 33, wherein the substrate has immobilized thereon at least ten different nucleic acids each comprising a different nucleotide sequence selected from the group consisting of SEQ ID NOs: 1-560 and complements thereof.

38. The substrate of claim 33, wherein the substrate has immobilized thereon at least twenty-five different nucleic acids each comprising a different nucleotide sequence selected from the group consisting of SEQ ID NOs: 1-560 and complements thereof.

39. The substrate of claim 33, wherein the substrate has immobilized thereon at least one hundred different nucleic acids each comprising a different nucleotide sequence selected from the group consisting of SEQ ID NOs: 1-560 and complements thereof.
